(19) United States
(12) Patent Application Publication
(10) Pub. No.: US 2011/0053851 A1
(43) Pub. Date: Mar. 3, 2011

(54) HAEMOSTASIS-MODULATING COMPOSITIONS AND USES THEREFOR

(75) Inventors: Paul Pantaleone Masci, Coorparoo (AU); John De Jersey, Chapel Hill (AU)

(73) Assignees: THE UNIVERSITY OF QUEENSLAND, St. Lucia (AU); VENOMICS PTY LTD, North Sydney (AU)

(21) Appl. No.: 12/808,356

(22) PCT Filed: Dec. 18, 2008
(86) PCT No.: PCT/AU08/01866

§ 371 Date: Nov. 19, 2010

Related U.S. Application Data
(60) Provisional application No. 61/015,587, filed on Dec. 20, 2007.

(51) Int. Cl. A61K 38/17; A61P 7/04

(52) U.S. Cl. 514/13.7

(57) ABSTRACT

This invention discloses the use of snake venom FV polypeptides in methods and compositions for preventing or reducing blood loss or bleeding during bleeding episodes.

### FIELD OF THE INVENTION

This invention relates generally to methods and agents for preventing or reducing blood loss or bleeding in a subject. More particularly, the present invention relates to administering to a subject a pharmaceutical composition comprising a snake venom FV, to prevent or reduce blood loss or bleeding during bleeding episodes.

### BACKGROUND OF THE INVENTION

Blood coagulation in response to vascular injury is vital for the continued survival of an organism. Given the importance of controlling blood loss during surgery or following injury or trauma, the identification of regulators that either promote blood clotting or inhibit the dissolution of clots (such as by the fibrinolytic plasmin/plasminogen pathway; Royston et al., 1990, Blood Coagul. Fibrinol. 1: 53; Orchard et al., 1993, Br. J. Haematol. 85: 596) has become an area of intense interest.

The coagulation process is mediated by a complex interaction of various blood components, or factors, which eventually gives rise to a fibrin clot. Generally, the blood components which participate in what has been referred to as the coagulation “cascade” are proenzymes or zymogens, enzymatically inactive proteins which are converted to proteolytic enzymes by the action of an activator, itself an activated clotting factor. Coagulation factors that have undergone such a conversion are generally referred to as “active factors”, and are designated by the addition of a lower case “a” suffix (e.g., Factor VIIa).

One of the key steps in the coagulation cascade is the conversion of prothrombin to thrombin by prothrombinase complex (FXa in complex with FVa), see Suttie, J. W and Jackson, C. M, 1977, Physiol Rev, 57: 1-70. There are two systems, or pathways, that promote the activation of Factor X (FX). The “intrinsic pathway” refers to those reactions that lead to thrombin formation through utilization of factors present only in plasma. A series of protease-mediated activations ultimately generates Factor IXa (FIXa) which, in conjunction with Factor VIIIa (FVIIIa), cleaves FX into FXa in the presence of Ca²⁺ and phospholipid. An identical proteolysis is effected by Factor VIIa (FVIIa) and its co-factor, tissue factor, in the “extrinsic pathway” of blood coagulation. Tissue factor is a membrane bound protein and does not normally circulate in plasma. Upon vessel disruption, however, it can complex with FVIIa to catalyse FX activation or Factor IX (FIX) activation in the presence of Ca²⁺ and phospholipid (Nemerson and Gentry, 1986, Biochem. 25: 4020-4033). While the relative importance of the two coagulation pathways in haemostasis is unclear, in recent years FVII and tissue factor have been found to play a pivotal role in the regulation of blood coagulation.

Factor VII is a trace plasma glycoprotein that circulates in blood as a single-chain zymogen. The zymogen is catalytically inactive (Williams et al., J. Biol. Chem. 264: 7536-7543 (1989); Rao et al., 1988, Proc. Natl. Acad. Sci. USA. 85: 6687-6691). Single-chain FVII may be converted to two-chain FVIIa by FXa, FXIIa, FIXa or thrombin in vitro. FXa is believed to be the major physiological activator of FVII. Like several other plasma proteins involved in haemostasis, FVII is dependent on vitamin K for its activity, which is required for the gamma-carboxylation of multiple glutamic acid residues that are clustered in the amino terminus of the protein. These gamma-carboxylated glutamic acids are required for the metal-associated interaction of FVII with phospholipids.

Factor V (FV) is a large glycoprotein synthesized as a single chain molecule and in mammals circulates in the blood as an inactive cofactor for the serine protease activated FX. When needed during injury or trauma, FV is proteolytically activated by thrombin or FXa. During activation, the B domain is released and the activated FVa is generated. FVa has two chains, a heavy chain (containing A1-A2 domains) and a light chain (containing A3-C1-C2 domains), which are held together by Ca²⁺ dependent non-covalent interactions.

Human FVa is inactivated proteolytically by activated protein C (APC) which provides an effective regulatory mechanism to maintain haemostatic balance. APC which is a vitamin K-dependent serine proteinase, cleaves human FVa at amino acid position 334, 534 and 679 which converts human FVa to inactive FV, thus destabilizing the prothrombinase complex, and reducing the rate of thrombin production.

For activation of prothrombin to take place at a physiologically relevant rate, FXa has to form the prothrombinase complex with FVa assembled on phospholipid membrane in the presence of Ca²⁺ ions (Suttie & Jackson, 1977, Physiol. Rev. 57: 1). The formation of this complex can boost the activation of prothrombin by 10⁵ fold as compared to the catalysis by FXa alone.

The Australian Brown Snake (Pseudonaja textilis and related species) and the two Taipans (Oxyuranus scutellus (coastal taipan) and Oxyuranus microlepidotus (inland taipan)) are unique in producing in their venoms, a potent procoagulant toxin made up of FXa-like protease in complex with a FVa-like cofactor, mimicking the human prothrombinase complex (Masci et al., 1988, Biochein Int, 17: 825-835). P. textilis FVa-like protease, lacks APC cleavage sites 334 and 534 and thus is not converted to its inactive form at the same rate as human FVa since it only has one cleavage site.

U.S. Pat. No. 7,125,846 discloses a method of treating bleeding episodes and coagulation disorders by administering human FV polypeptides combined with human FVII polypeptides either at the same time or one after the other. This administration is disclosed as providing a shortened clotting time, a firmer clot and an increased resistance to fibrinolysis compared to the clotting time, clot firmness and resistance when either FVIIa or FV is administered alone.

The present invention is predicated in part on the discovery that snake venom FVa proteins (e.g., P. textilis FVa protein) alone form a complex with human FXa to efficiently clot blood. This discovery is highly advantageous as the snake venom FVa protein will only form a prothrombinase complex (and clot blood) in the presence of FXa, which in humans is confined to the site of injury, allowing the snake venom FVa protein to be injected into a subject without clotting blood at undesirable sites. Illustrative examples of snake venom FV polypeptides include the following advantages: (1) the amount of snake venom FV protein required for maximum generation of thrombin from prothrombin is in the nM concentration range; (2) FV from snake venom requires only FXa to clot blood, whereas FVIIa requires tissue factor, Ca²⁺ and phospholipid; (3) the snake venom FV protein is extremely stable and not easy to degrade by APC since the snake venom FV amino acid sequence has less APC cleavage than the human FV sequence; (4) the concentration of the rate-limiting prothrombinase complex can be increased by the addition of snake venom FV to a much higher concentration than is achievable by the addition of FVIIa, since the endogenous FV is limiting; and (5) does not require snake venom FXa for activity. Not wishing to be bound by any one theory or mode of operation, it is proposed that since the concentration of FVa in mammalian blood is normally very low and the concentration of FV from which it is formed is also very low, the administration of snake venom FVa will increase the overall FVa concentration and hence increase the concentration of the prothrombinase complex. This in turn will substantially increase the rate of clot formation when required at the site of injury.

### SUMMARY OF THE INVENTION

Accordingly, in one aspect, the present invention provides methods for the treatment or prophylaxis of bleeding episodes or coagulation disorders in a subject. These methods generally comprise administering to the subject a bleeding-inhibiting effective amount of a snake venom FV polypeptide. In some embodiments, the snake venom FV polypeptide comprises: (a) an amino acid sequence that shares at least 50% (and at least 51% to at least 99% and all integer percentages in between) sequence similarity or sequence identity with the sequence set forth in any one of SEQ ID NO: 2, 4, 6, 8, 10 or 12; or (b) an amino acid sequence which is encoded by a nucleotide sequence that shares at least 50% (and at least 51% to at least 99% and all integer percentages in between) sequence similarity or sequence identity with the sequence set forth in any one of SEQ ID NO: 1, 3, 5, 7, 9 or 11, or a complement thereof; or (c) an amino acid sequence which is encoded by a nucleotide sequence that hybridizes under at least low, medium or high stringency conditions to the sequence set forth in any one of SEQ ID NO: 1, 3, 5, 7, 9 or 11, or a complement thereof, wherein the amino acid sequence of (a), (b) or (c) has any one or more activity selected from the group consisting of: bleeding-inhibitory activity, clotting time-reducing activity; haemostasis-enhancing activity; clot lysis time prolonging activity; and clot strength-increasing activity. Suitably, the snake venom FV polypeptide is administered in the form a composition comprising a pharmaceutically acceptable carrier.

In a related aspect, the present invention provides pharmaceutical compositions that comprise or consist essentially of a snake venom FV polypeptide as broadly defined herein and a pharmaceutically acceptable carrier. Suitably, the composition is formulated for systemic or local administration (e.g., topical or intravenous administration). In some embodiments, the composition excludes FVII and/or FVIIa. In some embodiments, the composition excludes snake venom FXa.

In some embodiments, the snake venom FV polypeptide comprises a light chain and a heavy chain domain, as shown for example in FIG. 7. Suitably, an activation peptide is interposed between the light chain and heavy chain domains, as shown for example in FIG. 7. In specific embodiments, however, this activation peptide is absent from the snake venom FV polypeptide.

In some embodiments the snake venom FV polypeptide includes one or more of: (a) a multicopper oxidase domain within the heavy chain region; (b) a multicopper oxidase domain within the light chain region; and (c) a C-terminal membrane-binding domain typically within the light chain region. In illustrative examples of this type, the snake venom FV polypeptide includes: (i) at least one (e.g., 2) multicopper oxidase domain within the heavy chain region; (ii) a multicopper oxidase domain within the light chain region; and (c) at least one (e.g., 2) membrane-binding domain within the light chain region.

In some embodiments the snake venom FV polypeptide includes one or more of: (1) a cupredoxin domain, as defined for example in the European Bioinformatics Institute (EBI) database under InterPro signature IPR008972 and Superfamily entry SSF49503; (2) a multicopper oxidase type 1 (copper binding site) domain (also referred to herein as a multicopper_oxidase 1 domain), as defined for example in the EBI InterPro database under InterPro signature IPR002355 and Prosite entry PS00079; (3) a multicopper oxidase type 2 domain (also referred to herein as a Cu-oxidase_{—}2 domain), as defined for example in the EBI database under InterPro signature IPRO11706 and Pfam entry PF07731; (4) a coagulation factor ⅝ type C domain (also referred to herein as a FA58C domain), as defined for example in the EBI database under InterPro signature IPR000421 and SMART Accession No. SM00231; (5) a coagulation factor ⅝ C-terminal domain (also referred to herein as a FA58C_{—}3 domain), as defined for example in the EBI database under InterPro signature IPR000421 and Prosite entry PS50022; (6) a coagulation factor ⅝ type C domain (also referred to herein as a F5_F8_type_C domain), as defined for example in the EBI database under InterPro signature IPR000421 and Pfam entry PF00754, (7) a galactose-binding-like domain (also referred to herein as a Gal_bind_like domain), as defined for example in the EBI database under InterPro signature IPR008979 and Superfamily entry SSF49785; (8) a coagulation factor ⅝ type C signature 1 domain (also referred to herein as a FA58C_{—}1 domain), as defined for example in the EBI database under InterPro signature IPR000421 and Prosite entry PS01285; (9) a coagulation factor ⅝ type C signature 2 domain (also referred to herein as a FA58C_{—}2 domain), as defined for example in the EBI database under InterPro signature IPR000421 and Prosite entry PS01286; and (10) a coagulation factor V domain (also referred to herein as a Factor_V domain), as defined for example in the EBI database under InterPro signature IPRO014693 and Protein Information Resource entry PIRSF5000150; or biologically active fragments thereof. In illustrative examples of this type, the snake venom FV polypeptide comprises: (1) at least one cupredoxin domain (e.g., 1, 2, 3, 4, 5 or 6 domains); (2) at least one multicopper_oxidase1 domain (e.g., 1, 2 or 3 domains); (3) a Cu-oxidase_{—}2 domain; (4) at least one FA58C domain (e.g., 1 or 2 domains); (5) at least one FA58C_{—}3 domain (e.g., 1 or 2 domains); (6) at least one F5_F8_type_C domain (e.g., 1 or 2 domains), (7) at least one Gal_bind_like domain (e.g., 1 or 2 domains); (8) at least one FA58C_{—}1 domain (e.g., 1 or 2 domains); (9) at least one FA58C_{—}2 domain (e.g., 1 or 2 domains); and (10) a Factor_V domain. In some embodiments, the snake venom FV polypeptide includes a signal peptide domain. In others, it lacks a signal peptide domain.

In some embodiments the snake venom FV polypeptide includes one or more of: (a) an A1 domain; (b) an A2 domain; (c) a B domain also referred to interchangeably as an activation domain; (d) an A3 domain; (e) a C1 domain; and (f) a C2 domain, wherein: the A1, A2 and A3 domains are diverged versions of the multicopper oxidase domain, as defined for example in the EBI database under InterPro signature IPRO01117; the B domain is removed during activation; and the C1 and C2 are membrane-binding promoting domains, as defined for example in the EBI database under InterPro signature IPRO00421. In illustrative examples of this type, the snake venom FV polypeptide comprises the A1, A2 and A3 domains as well as the C1 and C2 domains.

Representative domains are the same or very similar (differing, e.g., by 1, 2, 3, 4, 5 or even 10 residues) in length as the domains of naturally occurring species. In certain embodiments, the snake venom FV polypeptide comprises one or more (and in some cases all) of the following domains (the numbering refers to the consensus numbering in FIG. 7):
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, the heavy chain domain as defined for example by residues 31-772 of any of the snake venom FV polypeptides (also referred to hering as “snake venom FVs”) shown in FIG. 7;
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, the activation domain, also referred to interchangeably as the B domain, as defined for example by residues 773-817 of any of the snake venom FVs shown in FIG. 7;
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, the light chain domain as defined for example by residues 818-1461 of any of the snake venom FVs shown in FIG. 7;
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, any one of the cupredoxin domains as defined for example by residues 31-208, 209-337, 351-530, 500-682, 823-997 and 963-1153 of any of the snake venom FVs shown in FIG. 7;
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, any one of the multicopper_oxidase1 domains as defined for example by residues 307-327, 662-681 and 1120-1138 of any of the snake venom FVs shown in FIG. 7;
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, the Cu-oxidase_{—}2 domain as defined for example by residues 581-687 of any of the snake venom FVs shown in FIG. 7;
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, any one of the Gal_bind_like domains as defined for example by residues 1147-1298 and 1303-1457 of any of the snake venom FVs shown in FIG. 7;
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, any one of the FA58C domains as defined for example by residues 1146-1296 and1302-1455 of any of the snake venom FVs shown in FIG. 7;
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, any one of the FA58C_{—}3 domains as defined for example by residues 1147-1296 and 1303-1455 of any of the snake venom FVs shown in FIG. 7;
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, the FA58C_{—}2 domain as defined for example by residues 1141-1455 of any of the snake venom FVs shown in FIG. 7;
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, any one of the FA58C_{—}1 domains as defined for example by residues 1187-1215 and 1347-1374 of any of the snake venom FVs shown in FIG. 7; and
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, any one of the F5_F8_type_C domains as defined for example by residues 1162-1293 and 1318-1452 of any of the snake venom FVs shown in FIG. 7.

In some embodiments, the snake venom FV polypeptide comprises one or more (and in some cases all) of the following domains (the numbering refers to the consensus numbering in FIG. 7):
a domain which shares at least 70, 80, 85, 90, 91, 92, 93, 94, 95, 56, 97 or 98% sequence similarity or sequence identity with, or differs at no more than 1, 2, 3, 5 or even 10, 15 or 20 amino acid residues from, the A1 domain as defined for example by residues 31-348 of any of the snake venom FVs shown in FIG. 7;
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, the A2 domain as defined for example by residues 349-691 of any of the snake venom FVs shown in FIG. 7;
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, the B domain as defined for example by residues 772-817 of any of the snake venom FVs shown in FIG. 7;
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, the A3 domain as defined for example by residues 819-1148 of any of the snake venom FVs shown in FIG. 7;
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, the C1 domain as defined for example by residues 1149-1299 of any of the snake venom FVs shown in FIG. 7; and
a domain which shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, the C2 domain as defined for example by residues 1300-1461 of any of the snake venom FVs shown in FIG. 7.

In some embodiments, the activation or B domain is absent.

In specific embodiments, the heavy chain comprises the sequence:

[SEQ ID NO: 15]
AQLREYX₁X₂AAQLEDWDYNPQPEELSRLSESX₃LTFKKIVYREYELDFK
QEX₄X₅RDX₆LSGLLGPTLRGEVGDX₇LIIYFKNFATQPVSIHPQSAVYN
KWSEGSSYSDGTSDVERLDDAVPPGQSFKYVWNITAEIGPKKADPPCLTY
AYYSHVNMVRDFNSGLIGALLICKEGSLNAX₈GX₉QKFFNREYVLX₁₀FS
VFDESKNWYRKPSLQYTINGFANGTLPDVQACAYDHISWHLIGMSSSPEI
FSVHFNGQTLEQNHYKVSTIX₁₁X₁₂VGGASVTAX₁₃MSVSRTGKWLISS
LVAKHLQAGMYGYLNIKDCGX₁₄PX₁₅TLTRKLSFREX₁₆X₁₇X₁₈IX₁₉
X₂₀WEYFIAAEEITWDYX₂₁PEIPSSVDRRYKAQYLDNFSNFIGKKYKKA
VFRQYEDX₂₂NFTKPTYAIWPKERGILGPVIKAKVRDTVTIVFKNLASRP
YSIYVHGVSVSKDAEGAX₂₃YPSDPKENITHGKAVEPGQVYTYKWTVLDT
DEPTVKDSECITKLYHSAVDMTRDIASGLIGPLLX₂₄CKX₂₅KALSX₂₆
X₂₇GVQNKADVEQHAVFAVFDENKSWYLEDNIKKYCSNPSX₂₈VKKDDPK
FYKSNVMYTLNGYASDRTEVX₂₉X₃₀FHQSEVVX₃₁WHLTSVGTVDEIVP
VHLSGHTFLSKGKHQDILNLFPMSGESATVTMDNLGTWLLSSWGSCEMSN
GMRLRFLDANYDDEDEGNEEEEEDDGDIFADIFX₃₂PX₃₃EVVX₃₄KKEE
VPVNFVPDPESDALAKELGLX₃₅DDEX₃₆NPX₃₇X₃₈QX₃₉RX₄₀EQTED
DEEQLMX₄₁ASX₄₂LGLR,

wherein:

X₁ is selected from basic amino acid residues (e.g., Arg or His, or modified forms thereof);

X₂ is selected from hydrophobic amino acid residues (e.g., amino acid residues with an aliphatic side chain such as Ile or Leu, or modified forms thereof);

X₃ is selected from acidic amino acid residues (e.g., Glu or Asp, or modified forms thereof);

X₄ is selected from charged amino acid residues (e.g., basic amino acid residues such as Lys, or modified forms thereof, or acidic amino acid residues such Glu or modified forms thereof);

X₅ is selected from any amino acid residue (e.g., hydrophobic amino acid residues including ones with aliphatic side chains such as Leu, or modified forms thereof, or small amino acid residues such as Pro or modified forms thereof);

X₆ is selected from any amino acid residue (e.g., acidic amino acid residues such as Glu, or modified forms thereof, or small amino acid residues such Ala or modified forms thereof);

X₇ is selected from any amino acid residue (e.g., hydrophobic amino acid residues including ones with aliphatic sides chain such as Ile, or modified forms thereof, or small amino acid residues such as Ser, or modified forms thereof);

X₈ is selected from any amino acid residue (e.g., neutral/polar amino acid residues such as Asn, or modified forms thereof, or acidic amino acid residues such as Asp, or modified forms thereof);

X₉ is selected from small amino acid residues (e.g., Ser or Ala, or modified forms thereof);

X₁₀ is selected from hydrophobic amino acid residues (e.g., amino acid residues with aliphatic side chains such as Met or Val, or modified forms thereof);

X₁₁ is selected from any amino acid residue (e.g., acidic amino acid residues such as Asp, or modified forms thereof, or neutral/polar amino acid residues such as Asn, or modified forms thereof);

X₁₂ is selected from any amino acid residue (e.g., small amino acid residues such as Pro, or modified forms thereof, or hydrophobic amino acid residues including ones with an aliphatic side chain such as Leu, or modified forms thereof);

X₁₃ is selected from any amino acid residue (e.g., neutral/polar amino acid residues such as Asn, or modified forms thereof, or acidic amino acid residues such as Asp, or modified forms thereof);

X₁₄ is selected from any amino acid residue (e.g., basic amino acid residues such as His, or modified forms thereof, or neutral/polar amino acid residues such as Asn, or modified forms thereof);

X₁₅ is selected from any amino acid residue (e.g., neutral/polar amino acid residues such as Asn, or modified forms thereof, or acidic amino acid residues such as Asp, or modified forms thereof);

X₁₆ is selected from hydrophobic amino acid residues (e.g., an amino acid residue with an aliphatic side chain such as Leu, or modified forms thereof, or an amino acid residue with an aromatic side chain such as Trp, or modified forms thereof);

X₁₇ is selected from any amino acid residue (e.g., hydrophobic amino acid residues including ones with a sulfur-containing side chain such as Met, or modified forms thereof; or basic amino acid residues such as Arg, or modified forms thereof);

X₁₈ is selected from basic amino acid residues (e.g., Arg or Lys, or modified forms thereof);

X₁₉ is selected from any amino acid residue (e.g., hydrophobic amino acid residues including ones with a sulfur-containing side chain such as Met, or modified forms thereof; or basic amino acid residues such as Lys, or modified forms thereof);

X₂₀ is selected from any amino acid residue (e.g., neutral/polar amino acid residues such as Asn, or modified forms thereof, or basic amino acid residues such as Lys, or modified forms thereof);

X₂₁ is selected from small amino acid residues (e.g., Pro or Ala, or modified forms thereof);

X₂₂ is selected from small amino acid residues (e.g., Gly or Ser, or modified forms thereof);

X₂₃ is selected from hydrophobic amino acid residues (e.g., an amino acid residue with an aliphatic side chain such as Ile or Val, or modified forms thereof);

X₂₄ is selected from any amino acid residue (e.g., hydrophobic amino acid residues including ones with an aliphatic side chain, or modified forms thereof, or small amino acid residues such as Ala, or modified forms thereof);

X₂₅ is selected from any amino acid residue (e.g., hydrophobic amino acid residues including ones with an aliphatic side chain such as Leu, or modified forms thereof; or basic amino acid residues such as Arg or His, or modified forms thereof);

X₂₆ is selected from hydrophobic amino acid residues (e.g., an amino acid residue with an aliphatic side chain such as Ile or Val, or modified forms thereof);

X₂₇ is selected from basic amino acid residues (e.g., Arg or Lys, or modified forms thereof);

X₂₈ is selected from small amino acid residues (e.g., Ser or Ala, or modified forms thereof);

X₂₉ is selected from hydrophobic amino acid residues (e.g., an amino acid residue with an aliphatic side chain such as Leu, or modified forms thereof, or an amino acid residue with an aromatic side chain such as Trp, or modified forms thereof);

X₃₀ is selected from any amino acid residue (e.g., small amino acid residues such as Gly, or modified forms thereof, or basic amino acid residues such as Arg, or modified forms thereof);

X₃₁ is selected from any amino acid residue (e.g., neutral/polar amino acid residues such as Gln, or modified forms thereof, or acidic amino acid residues such as Glu, or modified forms thereof);

X₃₂ is selected from any amino acid residue (e.g., small amino acid residues such as Ser or modified forms thereof, or neutral/polar amino acid residues such as Asn, or modified forms thereof, or hydrophobic amino acid residues including ones with an aliphatic side chain such as Ile, or modified forms thereof);

X₃₃ is selected from small amino acid residues (e.g., Pro or Ser, or modified forms thereof);

X₃₄ is selected from any amino acid residue (e.g., basic amino acid residues such as Lys, or modified forms thereof, or hydrophobic amino acid residues including ones with an aliphatic side chain such as Ile, or modified forms thereof);

X₃₅ is selected from hydrophobic amino acid residues (e.g., an amino acid residue with an aliphatic side chain such as Leu or Ile, or modified forms thereof, or an amino acid residue with an aromatic side chain such as Phe, or modified forms thereof);

X₃₆ is selected from any amino acid residue (e.g., acidic amino acid residues such as Asp, or modified forms thereof, or small amino acid residues such as Gly, or modified forms thereof);

X₃₇ is absent or is selected from hydrophobic amino acid residues (e.g., ones with an aliphatic side chain such as Ile, or modified forms thereof);

X₃₈ is selected from any amino acid residue (e.g., charged amino acid residues including basic amino acid residues such as Lys, or modified forms thereof, and acidic amino acid residues such as Glu, or modified forms thereof, or hydrophobic amino acid residues including ones with an aliphatic side chain such as Ile, or modified forms thereof);

X₃₉ is selected from small amino acid residues (e.g., Ser or Pro, or modified forms thereof);

X₄₀ is selected from any amino acid residue (e.g., small amino acid residues such as Ser, or modified forms thereof, or basic amino acid residues such as Arg, or modified forms thereof);

X₄₁ is selected from any amino acid residue (e.g., hydrophobic amino acid residues including ones with an aliphatic side chain such as Ile, or modified forms thereof, or basic amino acid residues such as Lys, or modified forms thereof); and

X₄₂ is selected from hydrophobic amino acid residues (e.g., an amino acid residue with an aliphatic side chain such as Val, or modified forms thereof, or one with a sulfur-containing side chain such as Met, or modified forms thereof).

Representative embodiments of the activation peptide comprise the sequence:

[SEQ ID NO: 16]
SFKGSVAEEELKHTALALEEDAHASDPRIDSNSAX₄₃NX₄₄DDIAGRYL,

wherein:

X₄₃ is selected from basic amino acid residues (e.g., Arg or His, or modified forms thereof); and

X₄₄ is selected from small amino acid residues (e.g., Ser or Pro, or modified forms thereof).

In some embodiments, the light chain comprises the sequence:

[SEQ ID NO: 17]
RTIX₄₅RX₄₆NKRRYYIAAEEVLWDYSPIX₄₇KSQVRSX₄₈X₄₉AKTTFK
KAIFRSYLDDTFQTPSTGGEYEKHLGILGPIIRAEVDDVX₅₀EX₅₁QF
X₅₂NLASRPYSLHAHGLLYEKSSEGRSYDDX₅₃SPELFKKDDAIMPNGTY
TYVWQVPPRSGPTDNTEKCKSWAYYSGVNPEKDIHSGLIGPILICQKGMI
DKYNRTIDIREFVLFFMVFDEEKSWYFPKSDKSTCEEKLIGVQX₅₄SX₅₅
HTFPAINGIPYQLQGLX₅₆MYKDENVHWHLLNMGGPKDX₅₇HVVNFHGQT
FTEEGREDNQLGVLPLLPGTFASIKMKPSKIGTWLLETEVGENQERGX₅₈
QALFTVIDKX₅₉CKLPMGLASGIIQDSQISASGHVX₆₀YWEPKLARLNNT
GX₆₁X₆₂NAWSIIKKEHEHPWIQIDLQRQVVITGIQTQGTVX₆₃LLX₆₄H
SYTVEYFVTYSX₆₅DGQNWITFKGRHSX₆₆TQMHFEGNSDGTTVKENHID
PPIIARYIRLHPTKFYNX₆₇PTFRIELLGCEVEGCSVPLGMESGAIKX₆₈
SEITASSYKKTWWSSWEPX₆₉LARLNLX₇₀GX₇₁TNAWQPX₇₂VNNKDQW
LQIDLQHLTKITSIITQGATSMTTX₇₃MYVKTFSIHYTDDNSTWX₇₄PYL
DVRTSMEKVFTGNINX₇₅DGHVKHFFX₇₆PPILSRFIRIIPKTWNQYIAL
RIELFGCEVF,

wherein:

X₄₅ is selected from any amino acid residue (e.g., hydrophobic amino acid residues including ones with an aromatic side chain such as Tyr, or modified forms thereof, or neutral/polar amino acid residues such as Asn, or modified forms thereof);

X₄₆ is selected from any amino acid residue (e.g., basic amino acid residues such as Arg, or modified forms thereof, or small amino acid residues such as Gly, or modified forms thereof);

X₄₇ is selected from any amino acid residue (e.g., basic amino acid residues such as Arg, or modified forms thereof, or small amino acid residues such as Gly, or modified forms thereof);

X₄₈ is selected from any amino acid residue (e.g., hydrophobic amino acid residues including ones with an aliphatic side chain such as Leu, or modified forms thereof, or basic amino acid residues such as Arg, or modified forms thereof);

X₄₉ is selected from small amino acid residues (e.g., Pro or Ala, or modified forms thereof);

X₅₀ is selected from any amino acid residue (e.g., hydrophobic amino acid residues or modified forms thereof such as Ile, or modified forms thereof, or small amino acid residues such as Thr, or modified forms thereof;

X₅₁ is selected from hydrophobic amino acid residues (e.g., an amino acid residue with an aliphatic side chain such as Val or Ile, or modified forms thereof);

X₅₂ is selected from basic amino acid residues (e.g., Arg or Lys, or modified forms thereof);

X₅₃ is selected from any amino acid residue (e.g., neutral/polar amino acid residues such as Asn, or modified forms thereof, or basic amino acid residues such as Lys, or modified forms thereof);

X₅₄ is absent or is selected from small amino acid residues (e.g., Ser, or modified forms thereof);

X₅₅ is selected from is selected from any amino acid residue (e.g., basic amino acid residues such as Arg or His, or modified forms thereof, or hydrophobic amino acid residues including ones with an aliphatic side chain such as Leu, or modified forms thereof);

X₅₆ is selected from any amino acid residue (e.g., small amino acid residues such as Thr, or modified forms thereof, or hydrophobic amino acid residues including ones with a sulphur-containing side chain such as Met, or modified forms thereof);

X₅₇ is selected from any amino acid residue (e.g., hydrophobic amino acid residues including ones with an aliphatic side chain such as Ile, or modified forms thereof, or small amino acid residues such as Thr, or modified forms thereof);

X₅₈ is selected from any amino acid residue (e.g., hydrophobic amino acid residues including ones with a sulfur-containing side chain such as Met, or modified forms thereof, or small amino acid residues such as Thr, or modified forms thereof);

X₅₉ is selected from any amino acid residue (e.g., acidic amino acid residues such as Asp, or modified forms thereof, or small amino acid residues such as Gly, or modified forms thereof);

X₆₀ is selected from any amino acid residue (e.g., acidic amino acid residues such as Glu, or modified forms thereof, or small amino acid residues such as Gly, or modified forms thereof);

X₆₁ is selected from any amino acid residue (e.g., hydrophobic amino acid residues including ones with a sulfur-containing side chain such as Met, or modified forms thereof, or basic amino acid residues such as Lys, or modified forms thereof);

X₆₂ is selected from hydrophobic amino acid residues (e.g., an amino acid residue with an aromatic side chain such as Phe or Tyr, or modified forms thereof);

X₆₃ is selected from any amino acid residues (e.g., basic amino acid residues such as His, or modified forms thereof, or neutral/polar amino acid residues such as Gln, or modified forms thereof;

X₆₄ is selected from any amino acid residue (e.g., basic amino acid residues such as Lys, or modified forms thereof, or neutral/polar amino acid residues such as Gln, or modified forms thereof);

X₆₅ is selected from charged amino acid residues (e.g., basic amino acid residues such as Lys, or modified forms thereof, or acidic amino acid residues such Glu or modified forms thereof);

X₆₆ is selected from charged amino acid residues (e.g., acidic amino acid residues such Glu or modified forms thereof, or basic amino acid residues such as Lys, or modified forms thereof);

X₆₇ is selected from any amino acid residue (e.g., small amino acid residues such Thr or modified forms thereof, or basic amino acid residues such as Arg, or modified forms thereof);

X₆₈ is selected from any amino acid residue (e.g., neutral/polar amino acid residues such as Asn, or modified forms thereof, or acidic amino acid residues such as Asp, or modified forms thereof);

X₆₉ is selected from any amino acid residue (e.g., hydrophobic amino acid residues including ones with an aromatic side chain such as Phe, or modified forms thereof, or small amino acid residues such as Ser, or modified forms thereof);

X₇₀ is selected from charged amino acid residues (e.g., acidic amino acid residues such as Glu or modified forms thereof, or basic amino acid residues such as Lys, or modified forms thereof);

X₇₁ is selected from any amino acid residue (e.g., small amino acid residues such Gly or modified forms thereof, or basic amino acid residues such as Arg, or modified forms thereof);

X₇₂ is selected from charged amino acid residues (e.g., acidic amino acid residues such Glu or modified forms thereof, or basic amino acid residues such as Lys, or modified forms thereof);

X₇₃ is selected from small amino acid residues (e.g., Ala or Ser, modified forms thereof);

X₇₄ is selected from basic amino acid residues (e.g., Lys or Arg, or modified forms thereof);

X₇₅ is selected from small amino acid residues (e.g., Ser or Gly, modified forms thereof); and

X₇₆ is selected from any amino acid residue (e.g., basic amino acid residues such as Lys, or modified forms thereof, or neutral/polar amino acid residues such as Asn, or modified forms thereof).

In some embodiments, the snake venom FV polypeptide comprises a signal peptide, which suitably comprises the sequence: MGRYSVSPVPKCLLLMFLGWSGLKYYQ (SEQ ID NO: 18).

The snake venom FV polypeptides suitably encompass polypeptide sequences that comprise amino acid sequences that share at least 70, 80, 85, 90, 91, 92, 93, 94, 95, 96, 97 or 98% sequence similarity or sequence identity with, or differ at no more than 1, 2, 3, 5 or even 10, 15 or 20 amino acid residues from, the sequence set forth in any one of SEQ ID NO: 15, 16 and 17.

Representative snake venom FV polypeptides comprise a sequence that shares at least 70, 80, 85, 90, 91, 92, 93, 94, 95, 96, 97 or 98% sequence similarity or sequence identity with, or differs at no more than 1, 2, 3, 5 or even 10, 15 or 20 amino acid residues from, the sequence set forth in any one of SEQ ID NOs: 2 and 4 (brown snake), SEQ ID NOs: 6 and 8 (inland taipan) and SEQ ID NOs: 10 and 12 (coastal taipan). Suitably, the snake venom FV polypeptide lacks at least one of the signal peptide domain and the activation peptide (or B) domain.

In some embodiments, the snake venom FV polypeptide comprises any one or more of a FXa-binding site, a prothrombin-binding site and a thrombin cleavage site. In illustrative examples of this type, the snake venom FV polypeptide comprises at least one FXa-binding site (e.g., 1 or 2), a prothrombin-binding site and a thrombin cleavage site. Representative snake venom FV polypeptide comprise any one or more of:

(1) a FXa-binding site at about residues 338-379;

(2) a FXa-binding site at about residues 524-537;

(3) a prothrombin-binding site at about residues 703-707; and

(4) a thrombin cleavage site at about residues 772-773,

wherein the numbering refers to the consensus numbering in FIG. 7.

Suitably, the snake venom FV polypeptide comprises an APC site at residues 818-819 and/or residues 537-538 relative to the consensus numbering of FIG. 7. In some embodiments, the snake venom FV polypeptide has one or more (e.g., 1, 2, 3, 4 or 5) fewer Activated Protein C (APC) sites than a wild-type mammalian (e.g., human) FV.

In some embodiments, the snake venom FV polypeptide is administered in the form of a composition comprising a pharmaceutically acceptable carrier or diluent. The composition can be administered by injection (systemically) or by topical application to prevent or reduce blood loss in a subject, from the site of bleeding on or in the subject's body.

The composition can be used for the treatment of subjects experiencing bleeding episodes due to medical or surgical intervention, unwanted trauma, or other forms of tissue damage, illustrative examples of which include; coagulopathy, including coagulopathy in multi-transfused subjects; congenital or acquired coagulation or bleeding disorders, including decreased liver function (“liver disease”); defective platelet function or decreased platelet number; lacking or abnormal essential clotting “compounds” (e.g., platelets or von Willebrand factor protein); increased fibrinolysis; anticoagulant therapy or thrombolytic therapy; administration of a drug which reduces the ability of the subject to from or maintain a blood clot and; stem cell transplantation.

In some embodiments, the bleeding occurs in organs such as, for example the brain, inner ear region, eyes, liver, lung, tumor tissue, gastrointestinal tract; in other non-limiting examples, the bleeding is diffuse, such as for example in hemorrhagic gastritis and profuse uterine bleeding.

In other embodiments, the bleeding episodes are bleeding in connection with surgery or trauma in subjects having a deficiency in the ability to maintain or form a blood clot, for example due to acute haemarthroses (bleedings in joints), chronic haemophilic arthropathy, haematomas, (e.g., muscular, retroperitoneal, sublingual and retropharyngeal), bleeding in other tissue, haematuria (bleeding from the renal tract), cerebral haemorrhage, surgery (e.g., hepatectomy), dental extraction, and gastrointestinal bleedings (e.g., UGI bleeds). Furthermore, the composition can be used for treating bleeding episodes due to for example trauma, or surgery, or lowered count or activity of platelets, in a subject.

In another aspect, the invention relates to the use of a snake venom FV polypeptide as broadly defined above in the manufacture of a medicament or kit for preventing or reducing blood loss or bleeding in a subject. In some embodiments, the snake venom FV polypeptides is formulated for topical administration. The kit can comprise for example one or more of: a FV polypeptide in the form of a composition and a pharmaceutically acceptable carrier; one or more containers for the preparation of the snake venom FV polypeptide for administration to a subject; one or more other reagents and/or other therapeutic agents; devices or other materials for administering the snake venom FV polypeptide to a patient and; instructions for administering the kit to treat blood loss in a subject. In some embodiments, the kit excludes FVII and FVIIa. In some embodiments, the kit excludes snake venom FXa.

Suitably, the kit is used for reducing the time needed to obtain full haemostasis; reducing the time needed to maintain homeostasis; reducing clotting time; prolonging the clot lysis time; and increasing clot strength at the site of bleeding.

In some embodiments, the snake venom FV polypeptide is formulated for administration in an amount that is effective for achieving any one or more of the following: (1) inhibition of bleeding (i.e., a bleeding-inhibiting effective amount); (2) reduction of clotting time (i.e., clotting time-reducing effective amount); (3) enhancing hemostasis (i.e., haemostasis-enhancing effective amount); (4) prolonging clot lysis time (i.e., clot lysis time prolonging-effective amount); and (5) an increase in clot strength (i.e., clot strength-increasing effective amount).

In some embodiments, the snake venom FV polypeptide is formulated for administration by a person other than the subject. Alternatively, the snake venom FV polypeptide is formulated for self administration.

In some embodiments, the snake venom FV polypeptide is formulated for provision to the subject in advance of a need to use it.

### BRIEF DESCRIPTION OF THE DRAWINGS

FIG. 1 is a graphical representation of the clotting time of recalcified citrated plasma in the presence of phospholipid and P. textilis snake venom FV.

FIG. 2 is a graphical representation of the clotting time of citrated whole blood with increasing concentrations (0.4-550 nM) of P. textilis snake venom FV in the presence of added calcium. The control corresponds to a clotting time of 595 seconds (after recalcification of zero time).

FIG. 3 is a graphical representation showing a time course of blood loss in two groups, each of five mice. One group was treated topically with saline on the cut tail, as per the “Mouse Tail Excision Bleeding Model” described in paragraph [0256]. The second group was treated in the same way but with a solution of FVa in saline. Blood loss from each animal was measured in 10 minute time intervals and also in the treating solution.

FIG. 4 is a graphical representation using data from FIG. 3, showing: (a) the total blood loss in mice within each test group, with a total of five animals in each group (see the top graph) and; (b) the total average blood loss per 20 g mouse in each test group, with a total of five animals in each group (see the bottom graph).

FIG. 5 is a graphical representation of a mouse tail excision experiment after intravenous injection of 1000 of 500 nmol/L FVa in saline (▪); 100 μl of saline (control) (); and 100 μl of Aprotinin in saline (110 μmol/l) (▴). For each group, n=10.

FIG. 6 is a photographic representation of (a) a coomassie stained gel (gel 1) and (b) a Western blot (gel 2), which demonstrate that with the anti-protease heavy chain antibody (sheep antiserum against recombinant GST fusion protein with the heavy chain of P. textilis FXa-like protease), the affinity purified FV preparation appears depleted of Factor Xa. Lane 1 of gels (a) and (b) comprises P. textilis snake venom FV protein; Lane 2 comprises P. textilis FXa-like protease and; Lane 3 comprises FV (post Dardak and Xa affinity depletion).

FIG. 7 illustrates sequence alignments between isolated snake venom FV. Shown are amino acid sequences of snake venom FV derived from the following snakes: P. textilis (brown snake) (SEQ ID NO: 2), O. microlepidotus (inland taipan) (SEQ ID NOs: 6 and 8), and O. scutellatus (coastal taipan) (SEQ ID NOs: 10 and 12).

TABLE A
BRIEF DESCRIPTION OF THE SEQUENCES

| SEQUENCE ID | | |
|---|---|---|
| NUMBER | SEQUENCE | LENGTH |
| SEQ ID NO: 1 | Nucleotide sequence corresponding to the coding and | 4383 nts |
| | non-coding regions of the FV-like gene from | |
| | Pseudonaja textilis venom gland (AY168281). | |
| SEQ ID NO: 2 | FV-like polypeptide encoded by the coding region of | 1460 aa |
| | SEQ ID NO: 1. | |
| SEQ ID NO: 3 | Nucleotide sequence corresponding to the coding and | 4737 nts |
| | non-coding regions of the FV-like gene from | |
| | Pseudonaja textilis liver (AY576416). | |
| SEQ ID NO: 4 | FV-like polypeptide encoded by the coding region of | 1459 aa |
| | SEQ ID NO: 3. | |
| SEQ ID NO: 5 | Nucleotide sequence corresponding to the coding and | 4383 nts |
| | non-coding regions of the FV-like gene from Oxyuranus | |
| | microlepidotus venom gland (AY940210). | |
| SEQ ID NO: 6 | FV-like polypeptide encoded by the coding region of | 1460 aa |
| | SEQ ID NO: 5. | |
| SEQ ID NO: 7 | Nucleotide sequence corresponding to the coding and | 4380 nts |
| | non-coding regions of the FV-like gene from Oxyuranus | |
| | microlepidotus venom gland. | |
| SEQ ID NO: 8 | FV-like polypeptide encoded by the coding region of | 1459 aa |
| | SEQ ID NO: 7. | |
| SEQ ID NO: 9 | Nucleotide sequence corresponding to the coding and | 4380 nts |
| | non-coding regions of the FV-like gene from Oxyuranus | |
| | scutellatus venom gland (AY940209). | |
| SEQ ID NO: 10 | FV-like polypeptide encoded by the coding region of | 1459 aa |
| | SEQ ID NO: 9. | |
| SEQ ID NO: 11 | Nucleotide sequence corresponding to the coding and | 4675 nts |
| | non-coding regions of the FV-like gene from Oxyuranus | |
| | scutellatus venom gland. | |
| SEQ ID NO: 12 | FV-like polypeptide encoded by the coding region of | 1458 aa |
| | SEQ ID NO: 11. | |
| SEQ ID NO: 13 | Nucleotide sequence corresponding to the coding and | 9179 nts |
| | non-coding regions of the human FV gene. | |
| SEQ ID NO: 14 | Human FV polypeptide encoded by the coding region of | 2224 aa |
| | SEQ ID NO: 13. | |
| SEQ ID NO: 15 | Snake venom FV heavy chain | 742 aa |
| SEQ ID NO: 16 | Snake venom FV activation peptide | 45 aa |
| SEQ ID NO: 17 | Snake venom FV light chain | 644 aa |
| SEQ ID NO: 18 | Snake venom FV signal peptide | 27 aa |

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much 30, 25, 20, 25, 10, 9, 8, 7, 6, 5, 4, 3, 2 or 1% to a reference quantity, level, value, number, frequency, percentage, dimension, size, amount, weight or length.

The term “biologically active fragment”, as applied to fragments of a reference or full-length polynucleotide or polypeptide sequence, refers to a fragment that has at least about 0.1, 0.5, 1, 2, 5, 10, 12, 14, 16, 18, 20, 22, 24, 26, 28, 30, 35, 40, 45, 50, 55, 60, 65, 70, 75, 80, 85, 90, 95, 96, 97, 98, 99% of the activity of a reference sequence. Included within the scope of the present invention are biologically active fragments of at least about 18, 19, 20, 21, 22, 23, 24, 25, 26, 27, 28, 29, 30, 40, 50, 60, 70, 80, 90, 100, 120, 140, 160, 180, 200, 250, 300, 400, 500, 600, 700, 800, 900, 1000, 1500, 2000 nucleotides or residues in length, which comprise or encode an activity of a reference polynucleotide or polypeptide. Representative biologically active fragments generally participate in an interaction, e.g., an intramolecular or an inter-molecular interaction. An inter-molecular interaction can be a specific binding interaction or an enzymatic interaction. An inter-molecular interaction can be between a snake venom FV molecule and a FXa molecule. Biologically active portions of a snake venom FV protein include peptides comprising amino acid sequences with sufficient similarity or identity to or derived from the amino acid sequence of the snake venom FV of SEQ ID NO: 2, 4, 6, 8, 10 and 12.

The term “bleeding disorder” as used herein refers to any defect, congenital, acquired or induced, of cellular or molecular origin that is, manifested in bleeding episodes. Examples of bleeding disorders include but are not limited to clotting factor deficiencies, clotting factor inhibitors, defective platelet function, thrombocytopenia, von Willebrands disease, and coagulopathy such as that caused by dilution of coagulation proteins, increased fibrinolysis and lowered number of platelets due to bleeding and/or transfusions.

The term “bleeding episode” as used herein refers to unwanted, uncontrolled and often excessive bleeding in connection with surgery, trauma, or other forms of tissue damage, as well as unwanted bleeding in subjects having bleeding disorders as defined above.

The terms “clot lysis time”, “clot strength” and “clotting times” as used herein are clinical parameters used for assaying the status of a subject's haemostatic system. Blood samples can be drawn from the subject at suitable intervals and one or more parameters are assayed by means of for example, thromboelastography as described by, Meh et al., (2001, Blood Coagulation and Fibrinolysis, 12: 627-637); Vig et al., (2001, Hematology, 6(3): 205-213); Vig et al., (2001, Blood Coagulation and Fibrinolysis, 12(7): 555-561); Glidden et al., (2000, Clinical and Applied Thrombosis/Hemostasis, 6(4): 226-233; McKenzie et al., (1999, Cardiology, 92(4): 240-247) and; Davies et al., (1995, Journal of the American Society of Nephrology, 6(4): 1250-1255). The clot strength may be assayed as described in Carr et al., (1991, Am J Med, 302: 13-8).

The term “coagulation disorder” as used herein refers to disorders which disrupt the body's ability to control blood clotting. The most commonly known coagulation disorder is haemophilia, a condition in which patients bleed for long periods of time before clotting. There are other coagulation disorders with a variety of causes, non-limiting examples of which include; hemophilia B, hemophilia C, consumption coagulopathy, thrombocytopenia, Von Willebrands disease and hypoprothrombinemia.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “complementary” and “complementarily” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

By “effective amount”, in the context of treating or preventing a condition is meant the administration of that amount of active agent to an individual in need of such treatment or prophylaxis, either in a single dose or as part of a series, that is effective for treatment of, or prophylaxis against, that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As used herein, the terms “function” and “functional” and the like refer to a biological, enzymatic, or therapeutic function.

The term “FV polypeptide” as used herein encompasses, without limitation, snake venom FV polypeptides and is intended to encompass, without limitation, polypeptides having the amino acid sequence that shares at least 50% (and at least 51% to at least 99% and all integer percentages in between) sequence identity with the sequence set forth in any one of SEQ ID NOs: 2, 4, 6, 8, 10 or 12, as well as wild type (naturally-occurring) FV derived from other species, such as, e.g., bovine, porcine, canine, murine, rat and salmon. It further encompasses natural allelic variation of FV that may exist and occur from one individual to another. Also, degree and location of glycosylation or other post-translation modifications may vary depending on the chosen host and the nature of the hosts cellular environment. The term “FV” is also intended to encompass FV polypeptides in their zymogen form, as well as those that have been processed to yield their respective bioactive forms. It further encompasses FV polypeptides that have either been chemically modified relative to a reference or naturally-occurring snake venom FV and/or contain one or more amino acid sequence alterations relative to a reference or naturally-occurring snake venom FV and/or contain truncated amino acid sequences relative to a reference or naturally-occurring full-length or precursor snake venom FV. Thus, for example, snake venom FV polypeptides encompass processed forms of a naturally-occurring or reference full-length or precursor snake venom FV, including but not limited to FVa polypeptides. Alternatively, or in addition, snake venom FV polypeptides may exhibit different properties relative to a reference or naturally-occurring snake venom FV, including stability, phospholipid binding, altered specific activity and the like. The term “FV polypeptides” also encompasses polypeptides with a slightly modified amino acid sequence, for instance, polypeptides having modified N-terminal end including N-terminal amino acid deletions or additions, and/or polypeptides that have been chemically modified relative to a reference or naturally-occurring snake venom FV. FV polypeptides also encompass polypeptides exhibiting substantially the same or better bioactivity than a reference or naturally-occurring FV, or, alternatively, exhibiting substantially modified or reduced bioactivity relative to a reference or naturally-occurring FV. They also include, without limitation, polypeptides having an amino acid sequence that differs from the sequence of a reference or naturally-occurring FV by insertion, deletion, or substitution of one or more amino acids and in illustrative examples, encompass polypeptides that exhibit at least about 10%, 20%, 30%, 40%, 50%, 60%, 70%, 80%, 90%, 100%, 110%, 120%, and 130% of the specific activity of a reference or naturally-occurring FV that has been produced in the same cell. FV polypeptides having substantially the same or improved biological activity relative to a reference or naturally-occurring FV, encompass polypeptides that exhibit at least about 25%, 50%, 75%, 100%, 110%, 120% or 130% of the specific biological activity of the reference or naturally-occurring FV that has been produced in the same cell type. For purposes of the present invention, FV biological activity may be quantified, for example, by measuring the ability of a preparation to clot plasma (as described in Thorelli et al., 1998, Thromb Haemost, 80: 92). Conversely, FV polypeptides having substantially reduced biological activity relative to a reference or naturally-occurring FV are those that exhibit less than about 25%, 10%, 5% or 1% of the specific activity of the reference or naturally-occurring FV that has been produced in the same cell type.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

The term “haemostasis” as used herein refers to the formation of a stable and solid fibrin clot or plug at the site of injury which effectively stops the bleeding and which is not readily dissolved by the fibrinolytic system.

“Homology” refers to the percentage number of nucleic or amino acids that are identical or constitute conservative substitutions. Homology may be determined using sequence comparison programs such as GAP (Deveraux et al., 1984, Nucleic Acids Research 12, 387-395) which is incorporated herein by reference. In this way sequences of a similar or substantially different length to those cited herein could be compared by insertion of gaps into the alignment, such gaps being determined, for example, by the comparison algorithm used by GAP.

The term “host cell” includes an individual cell or cell culture which can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention is a recombinant host cell.

“Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

By “isolated” is meant material that is substantially or essentially free from components that normally accompany it in its native state. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell, i.e., it is not associated with in vivo substances.

By “obtained from” is meant that a sample such as, for example, a polynucleotide extract or polypeptide extract is isolated from, or derived from, a particular source of the subject. For example, the extract can be obtained from a tissue or a biological fluid isolated directly from the subject.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

The term “operably linked” as used herein means placing a structural gene under the regulatory control of a promoter, which then controls the transcription and optionally translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the genes from which it is derived.

The terms “patient” and “subject” are used interchangeably and refer to patients and subjects of human or other mammals and includes any individual it is desired to examine or treat using the methods of the invention. However, it will be understood that “patient” does not imply that symptoms are present. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes).

By “pharmaceutically acceptable carrier” is meant a solid or liquid filler, diluent or encapsulating substance that can be safely used in topical or systemic administration to an animal, preferably a mammal, including humans.

The term “polynucleotide” or “nucleic acid” as used herein designates mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. The terms “polynucleotide variant” and “variant” also include naturally occurring allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

The term “polypeptide variant” refers to polypeptides that are distinguished from a reference polypeptide by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotide residues, although it can contain fewer nucleotide residues. Primers can be large polynucleotides, such as from about 200 nucleotide residues to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide. Preferably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide”, through complementary base pairing. Probes can bind target polynucleotides lacking complete sequence complementarity with the probe, depending on the stringency of the hybridization conditions. Probes can be labeled directly or indirectly.

By “regulatory element” or “regulatory sequence” is meant nucleic acid sequences (e.g., DNA) necessary for expression of an operably linked coding sequence in a particular host cell. The regulatory sequences that are suitable for prokaryotic cells for example, include a promoter, and optionally a cis-acting sequence such as an operator sequence and a ribosome binding site. Control sequences that are suitable for eukaryotic cells include promoters, polyadenylation signals, transcriptional enhancers, translational enhancers, leader or trailing sequences that modulate mRNA stability, as well as targeting sequences that target a product encoded by a transcribed polynucleotide to an intracellular compartment within a cell or to the extracellular environment.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. The present invention contemplates the use in the methods and systems of the present invention of full-length snake venom FV sequences as well as their biologically active fragments. Typically, biologically active fragments of a full-length snake venom FV may participate in an interaction, for example, an intra-molecular or an inter-molecular interaction. An inter-molecular interaction can be a specific binding interaction or an enzymatic interaction (e.g., the interaction can be transient and a covalent bond is formed or broken). Biologically active fragments of a full-length snake venom FV include peptides comprising amino acid sequences sufficiently similar to or derived from the amino acid sequences of a (putative) full-length snake venom FV. Typically, biologically active fragments comprise a domain or motif with at least one activity of a full-length snake venom FV and may include one or more (and in some cases all) of a A1, A2, B, A3, C1 or C2 domain. A biologically active fragment of a full-length snake venom FV can be a polypeptide which is, for example, 30, 40, 50, 60, 70, 80, 90, 100, 120, 150, 300, 400 or 500, or more amino acid residues in length. Suitably, the biologically-active fragment has no less than about 1%, 10%, 25% 50% of an activity of the full-length polypeptide from which it is derived.

“Similarity” refers to the percentage number of amino acids that are identical or constitute conservative substitutions as defined in Table C infra. Similarity may be determined using sequence comparison programs such as GAP (Deveraux et al. 1984, Nucleic Acids Research 12: 387-395). In this way, sequences of a similar or substantially different length to those cited herein might be compared by insertion of gaps into the alignment, such gaps being determined, for example, by the comparison algorithm used by GAP.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

“Stringency” as used herein, refers to the temperature and ionic strength conditions, and presence or absence of certain organic solvents, during hybridization and washing procedures. The higher the stringency, the higher will be the degree of complementarity between immobilized target nucleotide sequences and the labeled probe polynucleotide sequences that remain hybridized to the target after washing. The term “high stringency” refers to temperature and ionic conditions under which only nucleotide sequences having a high frequency of complementary bases will hybridize. The stringency required is nucleotide sequence dependent and depends upon the various components present during hybridization. Generally, stringent conditions are selected to be about 10 to 20° C. lower than the thermal melting point (Tₘ) for the specific sequence at a defined ionic strength and pH. The Tₘ is the temperature (under defined ionic strength and pH) at which 50% of a target sequence hybridizes to a complementary probe.

The term “transformation” means alteration of the genotype of an organism, for example a bacterium, yeast, mammal, avian, reptile, fish or plant, by the introduction of a foreign or endogenous nucleic acid.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction site and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in animal and preferably mammalian cells. Such vector may be derived from a poxvirus, an adenovirus or yeast. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin®) and the hph gene which confers resistance to the antibiotic hygromycin B.

The terms “wild-type” and “naturally occurring” are used interchangeably to refer to a gene or gene product that has the characteristics of that gene or gene product when isolated from a naturally occurring source. A wild type gene or gene product (e.g., a polypeptide) is that which is most frequently observed in a population and is thus arbitrarily designed the “normal” or “wild-type” form of the gene.

### 2. FV Molecules of the Invention

The present invention is based in part on the determination that snake venom FV polypeptides when administered to a subject in the absence of other clotting factors such as factor Xa and/or factor VIIa can substantially enhance clotting of human plasma and whole blood. The inventors have surprisingly discovered that the addition of a low concentration of FV polypeptides (e.g., in the nanomolar range) from the venom of the Australian Brown snake, Pseudonaja textilis, alone can cause a significant increase in the rate of clotting of citrated plasma. The present inventors therefore consider that snake venom FV polypeptides will be useful in treating or preventing blood loss from wounds following surgery injury or trauma, as well as in individuals with intrinsically low levels of factor V in their blood (e.g., in individuals with parahemophilia or Owren parahemophilia whose symptoms may include any one or more of bleeding into the skin, excessive bruising, nose bleeds, bleeding of the gums, excessive menstrual bleeding, prolonged or excessive loss of blood with surgery or trauma, and umbilical stump bleeding).

Accordingly, the present invention provides methods for preventing or reducing blood loss or bleeding in a subject, wherein FV polypeptides are administered in the form of a composition that optionally comprises a pharmaceutically acceptable carrier or diluent. The composition can be administered by injection or by topical application to prevent or reduce blood loss in a subject. Non-limiting examples of snakes from which snake venom polypeptides can be obtained include the Australian common brown snake Pseudonaja textilis, coastal taipan (Oxyuranus scutellatus), inland taipan (Oxyuranus microlepidotus), mainland tiger (Notechis scutatus), rough scaled (Tropidechis carinatus) and red-belly black snake (Pseudechis porphyriacus) and other snakes from the genus Elapidae. Snake venom FV polypeptides can be isolated from snake venom or from other sources including cells and tissues that produce snake venom FV using standard protein purification techniques. In some embodiments, the snake venom FV is isolated from the venom gland of an Australian snake from the genus Elapidae, e.g., any one of the Australian snakes listed above. Alternatively, snake venom FV protein or fragments thereof can be produced by recombinant DNA techniques or synthesized chemically.

Polypeptides of the invention include those which arise as a result of the existence of alternative translational and post-translational events. The polypeptide can be expressed in systems, e.g., cultured cells, which result in substantially the same post-translational modifications present when expressed the polypeptide is expressed in a native cell, or in systems which result in the alteration or omission of post-translational modifications, e.g., glycosylation or cleavage, present when expressed in a native cell.

In some embodiments, a snake venom FV polypeptide has any one or more of the following characteristics:

it has the ability to clot blood when FXa (e.g., a mammalian FXa such as but not limited to human FXa) is present;

it has at least one multicopper oxidase domain (e.g., A1, A2, A3 domains);

it has at least one membrane-binding domain (e.g., C1, C2 domains);

it has a light and a heavy chain.

In some embodiments, a snake venom FV polypeptide is in the form of a single polypeptide chain, as described for example by Bos et al., (2007, Blood ASH Annual Meeting Abstracts 110: Abstract 1765).

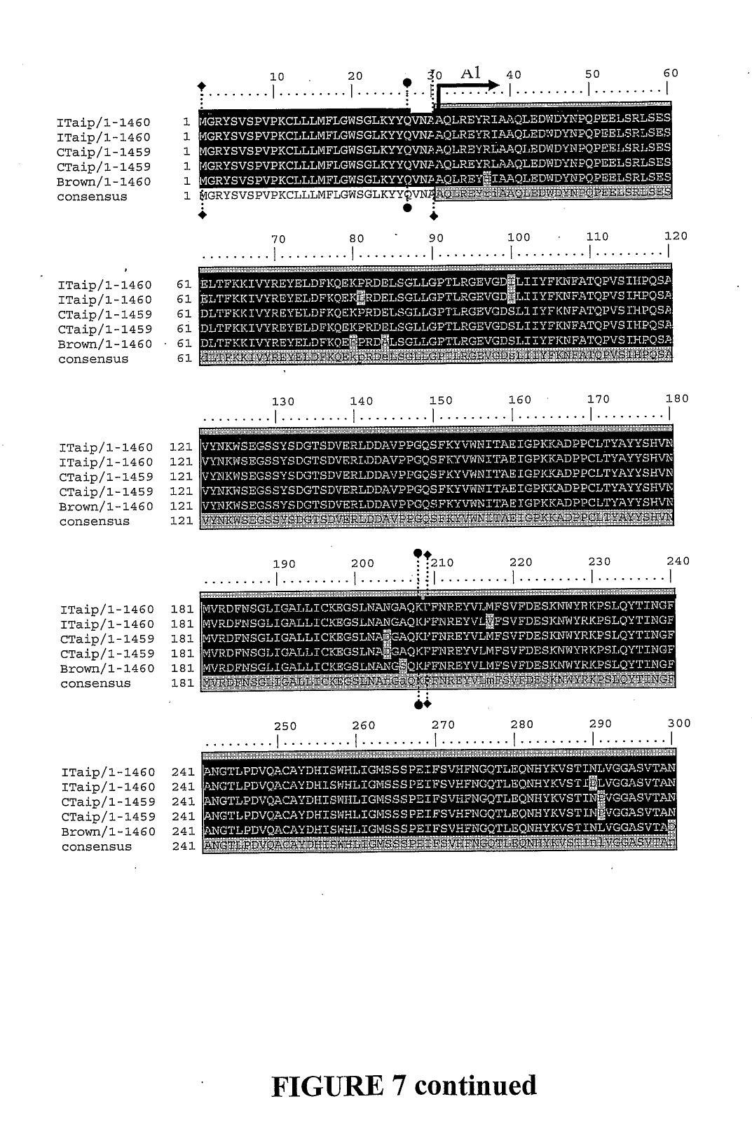

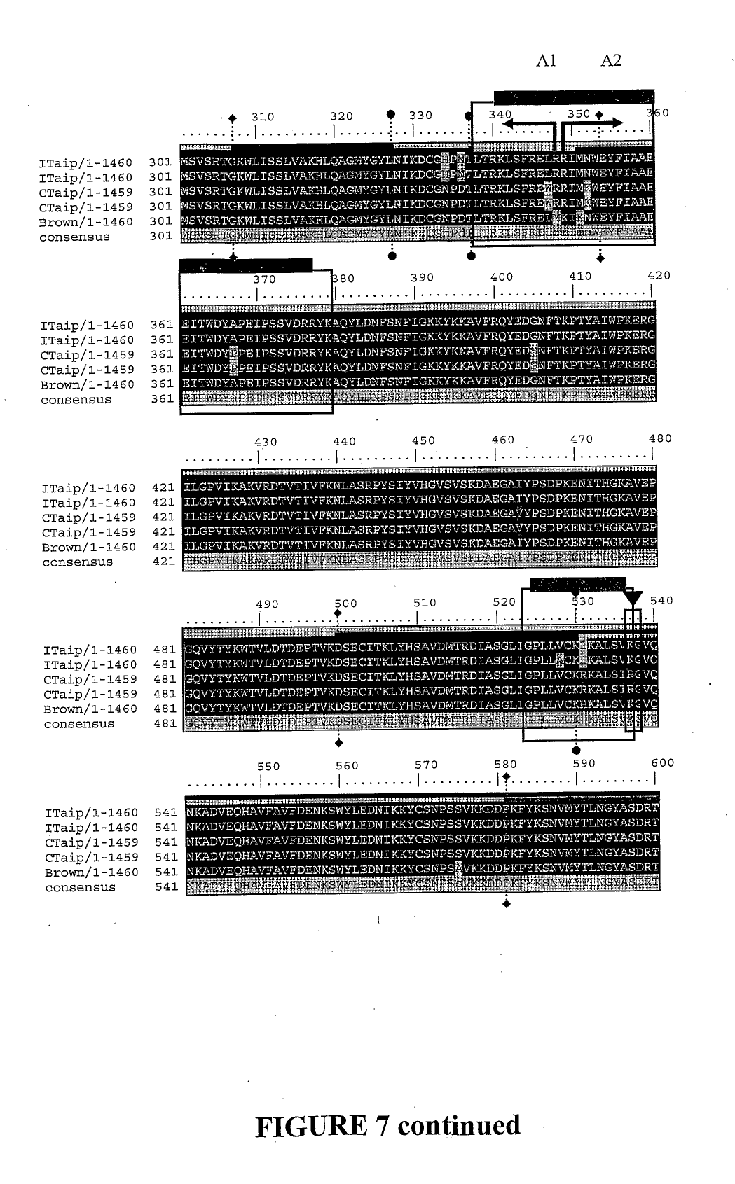

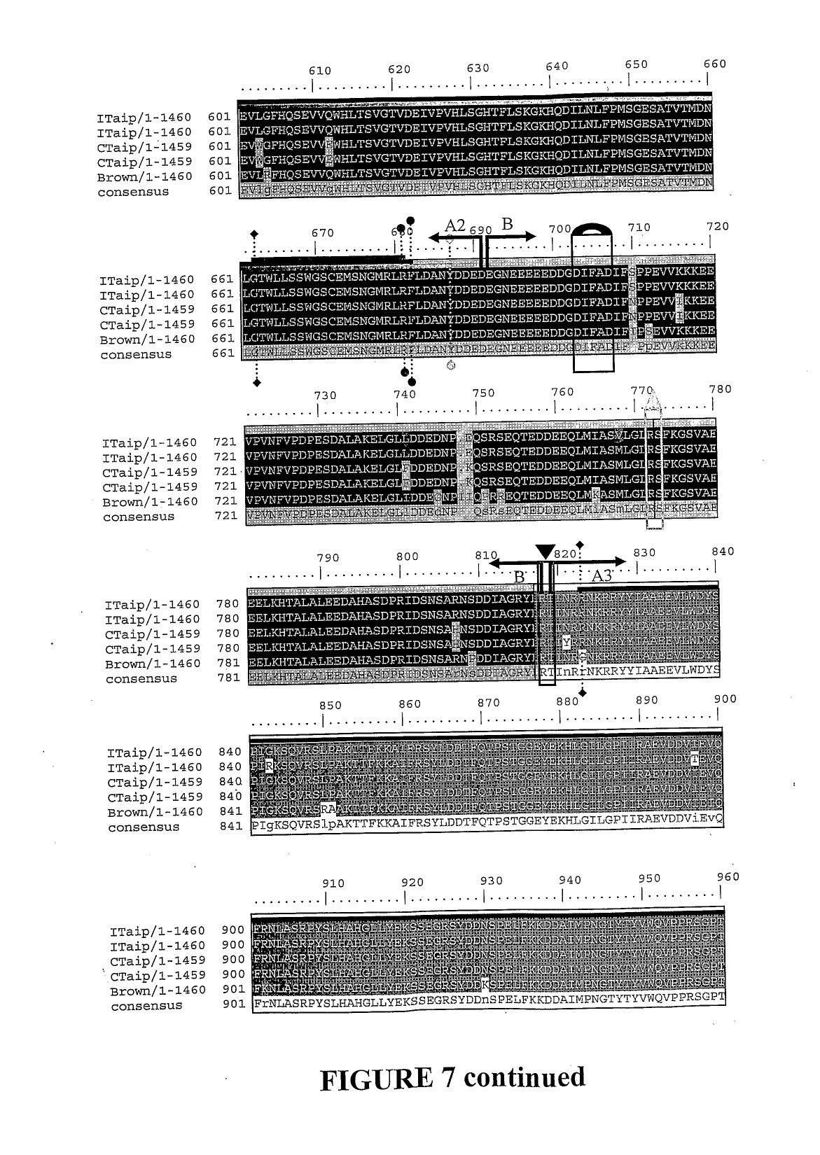

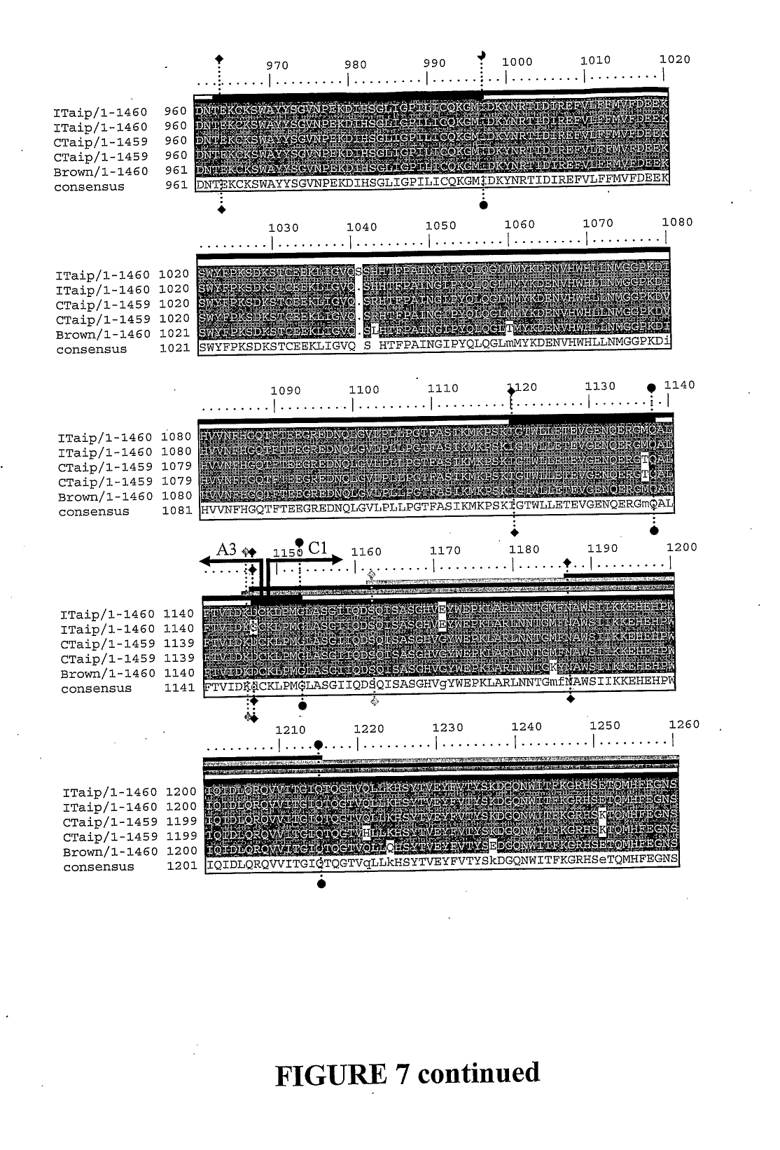

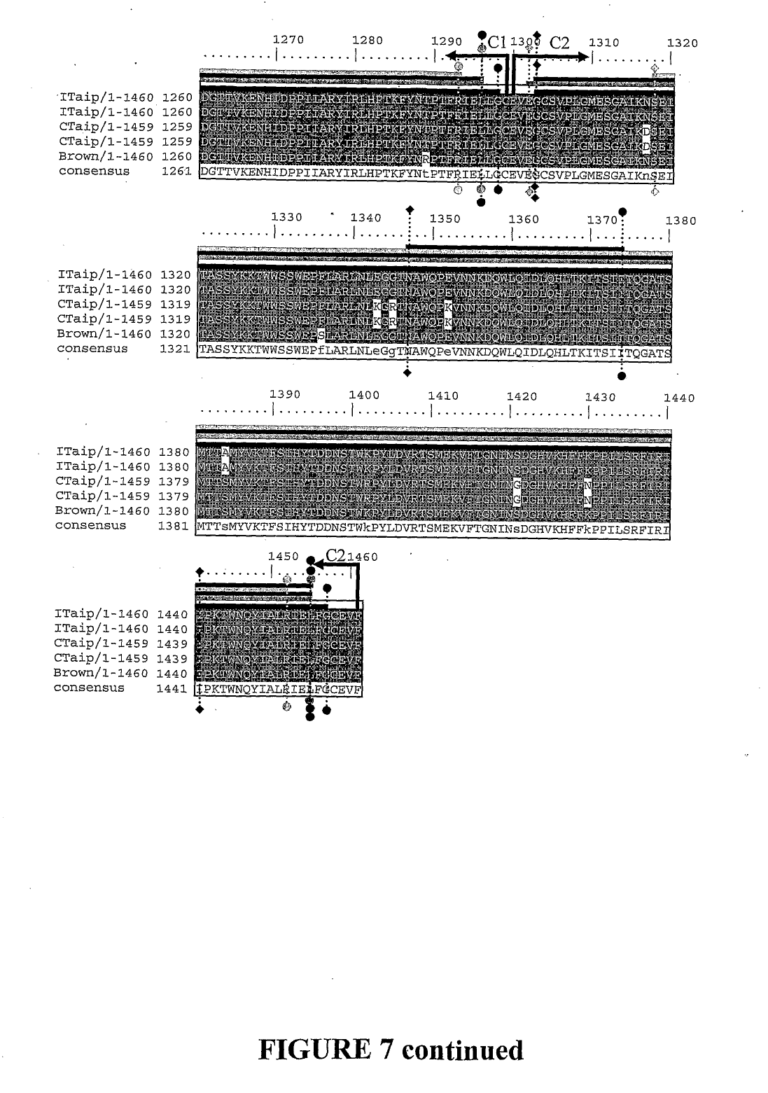

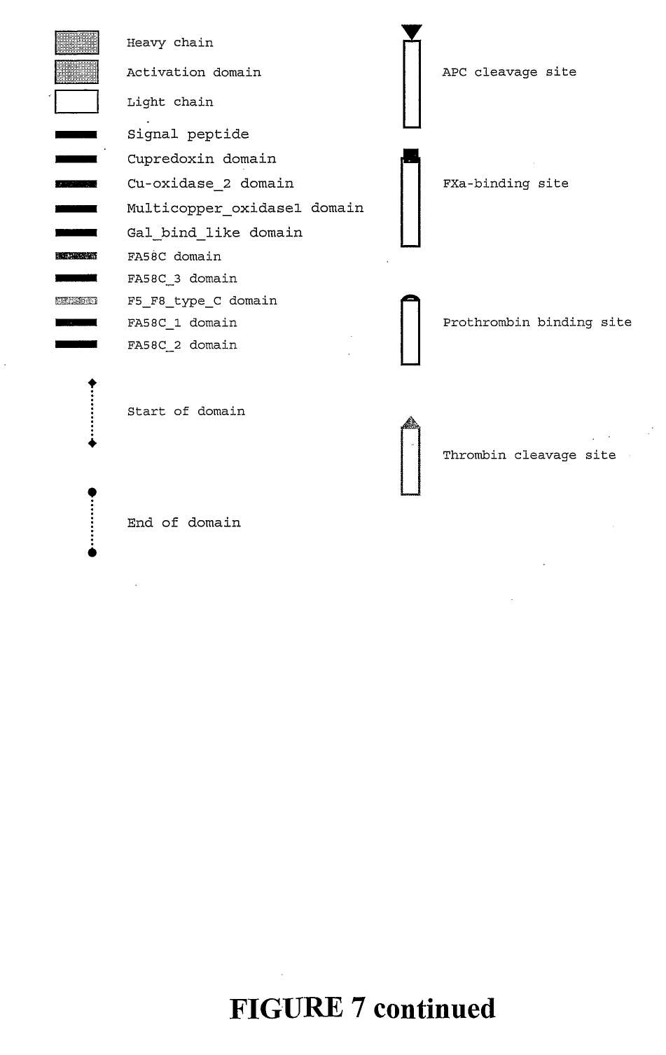

The present invention contemplates the use in the methods of the present invention of full-length snake venom FV polypeptides as well as their biologically active fragments. Typically, biologically active fragments of a full-length snake venom FV polypeptide may participate in an interaction, for example, an intramolecular or an inter-molecular interaction. Such biologically active fragments include peptides comprising amino acid sequences sufficiently similar to or derived from the amino acid sequences of a (putative) full-length snake venom FV polypeptide, for example, the amino acid sequences shown in SEQ ID NO: 2, 4, 6, 8, 10 or 12, which include less amino acids than a full-length snake venom FV polypeptide, and exhibit at least one activity of that polypeptide. Typically, biologically active fragments will comprise a domain or motif with at least one activity of a full-length snake venom FV polypeptide and may include one or more (and in some cases all) of a multicopper oxidase domain (e.g., type 1, type 2), a membrane-binding domain, a cupredoxin domain, a coagulation factor ⅝ type domain (e.g., FA58C, FA58C_{—}3, F5_F8_type_C, FA58C_{—}2, FA58C_{—}1), a galactose-binding-like domain, a FXa-binding site, a prothrombin-binding site, a thrombin cleavage site. In some embodiments, biologically active fragments will comprise one or more APC cleavage sites, for example at residues 818-819 and/or residues 537-538 relative to the consensus numbering of FIG. 7. A biologically active fragment of a full-length snake venom FV polypeptide can be a polypeptide which is, for example, 30, 40, 50, 60, 70, 80, 90, 100, 120, 150, 300, 400 or 500, 600, 800, 900, 1000, 1100, 1200, 1300, 1400, or more amino acid residues in length. Suitably, the biologically-active fragment has no less than about 1%, 10%, 25% 50% of an activity of the full-length polypeptide from which it is derived.

The present invention also contemplates snake venom FV polypeptides that are variants of wild-type or naturally-occurring snake venom FVs. Such “variant” polypeptides include proteins derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Non-limiting examples of such FV variant polypeptides include processed forms of a full-length or precursor snake venom FV, including but not limited to FVa polypeptides in which the signal peptide domain and activation peptide (or B) domain have been removed from the zymogen or precursor form. In some embodiments, the variant polypeptides have one or more (e.g., 1, 2, 3, 4 or 5) fewer Activated Protein C (APC) sites than a wild-type mammalian (e.g., human) FV.

Variant proteins encompassed by the present invention are biologically active, that is, they continue to possess the desired biological activity of the native protein. Such variants may result from, for example, genetic polymorphism or from human manipulation. Biologically active variants of a native or wild-type snake venom FVs will have at least 40%, 50%, 60%, 70%, generally at least 75%, 80%, 85%, usually about 90% to 95% or more, and typically about 98% or more sequence similarity or identity with the amino acid sequence for the native protein as determined by sequence alignment programs described elsewhere herein using default parameters. A biologically active variant of a wild-type snake venom FV polypeptide, which fall within the scope of a variant polypeptide, may differ from that protein generally by as much 200, 100, 50 or 20 amino acid residues or suitably by as few as 1-15 amino acid residues, as few as 1-10, such as 6-10, as few as 5, as few as 4, 3, 2, or even 1 amino acid residue. In some embodiments, a variant polypeptide differs from the corresponding sequences in SEQ ID NO: 2, 4, 6, 8, 10 and 12 by at least one but by less than 15, 10 or 5 amino acid residues. In other embodiments, it differs from the corresponding sequences in SEQ ID NO: 2, 4, 6, 8, 10 and 12 by at least one residue but less than 20%, 15%, 10% or 5% of the residues.

A snake venom FV polypeptide may be altered in various ways including amino acid substitutions, deletions, truncations, and insertions. Methods for such manipulations are generally known in the art. For example, amino acid sequence variants of a FV polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (“Molecular Biology of the Gene”, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.). Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of FV polypeptides. Recursive ensemble mutagenesis (REM), a technique which enhances the frequency of functional mutants in the libraries, can be used in combination with the screening assays to identify FV polypeptide variants (Arkin and Yourvan (1992) Proc. Natl. Acad. Sci. USA 89: 7811-7815; Delgrave et al., (1993) Protein Engineering, 6: 327-331). Conservative substitutions, such as exchanging one amino acid with another having similar properties, may be desirable as discussed in more detail below.

Variant FV polypeptides may contain conservative amino acid substitutions at various locations along their sequence, as compared to a parent (e.g., naturally-occurring or reference) FV amino acid sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al., (1978), A model of evolutionary change in proteins. Matrices for determining distance relationships In M. O. Dayhoff, (ed.), Atlas of protein sequence and structure, Vol. 5, pp. 345-358, National Biomedical Research Foundation, Washington D.C.; and by Gonnet et al., (1992, Science, 256(5062): 14430-1445), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behaviour.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always non-aromatic. Dependent on their structural properties, amino acid residues may fall in two or more classes. For the naturally-occurring protein amino acids, sub-classification according to this scheme is presented in Table B.

TABLE B
AMINO ACID SUB-CLASSIFICATION

| SUB-CLASSES | AMINO ACIDS |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional FV polypeptide can readily be determined by assaying its activity. Conservative substitutions are shown in Table C under the heading of exemplary and preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE C
EXEMPLARY AND PREFERRED AMINO ACID SUBSTITUTIONS

| ORIGINAL | EXEMPLARY | PREFERRED |
|---|---|---|
| RESIDUE | SUBSTITUTIONS | SUBSTITUTIONS |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm.C. Brown Publishers (1993).

Thus, a predicted non-essential amino acid residue in a snake venom FV polypeptide is typically replaced with another amino acid residue from the same side chain family. Alternatively, mutations can be introduced randomly along all or part of a snake venom FV gene coding sequence, such as by saturation mutagenesis, and the resultant mutants can be screened for an activity of the parent polypeptide to identify mutants which retain that activity. Following mutagenesis of the coding sequences, the encoded peptide can be expressed recombinantly and the activity of the peptide can be determined. A “non-essential” amino acid residue is a residue that can be altered from the wild-type sequence of an embodiment polypeptide without abolishing or substantially altering one or more of its activities. Suitably, the alteration does not substantially alter one of these activities, for example, the activity is at least 20%, 40%, 60%, 70% or 80% of wild-type. Illustrative non-essential amino acid residues include any one or more of the amino acid residues that differ at the same position (e.g., residues X₁-X₆₆, as defined supra) between the wild-type snake venom FV polypeptides shown in FIG. 7. An “essential” amino acid residue is a residue that, when altered from the wild-type sequence of a reference FV polypeptide, results in abolition of an activity of the parent molecule such that less than 20% of the wild-type activity is present. For example, such essential amino acid residues include those that are conserved in FV polypeptides across different species, e.g., W(N/D)Y(A/P)P that is conserved in the FXa-binding site of the FV polypeptides from human, mouse, bovine, chicken, brown snake, inland taipan and coastal taipan, which binding site is defined for example by residues 338-379, as shown in FIG. 7.

Accordingly, the present invention also contemplates as FV polypeptides, variants of the naturally-occurring FV polypeptide sequences or their biologically-active fragments, wherein the variants are distinguished from the naturally-occurring sequence by the addition, deletion, or substitution of one or more amino acid residues. In general, variants will display at least about 30, 40, 50, 55, 60, 65, 70, 75, 80, 85, 90, 91, 92, 93, 94, 95, 96, 97, 98, 99% similarity to a parent or reference FV polypeptide sequence as, for example, set forth in SEQ ID NO: 2, 4, 6, 8, 10 and 12. Desirably, variants will have at least 30, 40, 50, 55, 60, 65, 70, 75, 80, 85, 90, 91, 92, 93, 94, 95, 96, 97, 98, 99% sequence identity to a parent FV polypeptide sequence as, for example, set forth in SEQ ID NO: 2, 4, 6, 8, 10 and 12. Moreover, sequences differing from the native or parent sequences by the addition, deletion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100 or more amino acids but which retain the properties of the parent FV polypeptide are contemplated. FV polypeptides also include polypeptides that are encoded by polynucleotides that hybridize under stringency conditions as defined herein, especially high stringency conditions, to FV-encoding polynucleotide sequences, or the non-coding strand thereof, as described below. Illustrative snake venom FV polynucleotide sequences are set forth in SEQ ID NO: 1, 3, 5, 7, 9 and 11.

In some embodiments, variant polypeptides differ from a reference FV sequence by at least one but by less than 50, 40, 30, 20, 15, 10, 8, 6, 5, 4, 3 or 2 amino acid residue(s). In other embodiments, variant polypeptides differ from the corresponding sequences of SEQ ID NO: 2, 4, 6, 8, 10 and 12 by at least 1% but less than 20%, 15%, 10% or 5% of the residues. (If this comparison requires alignment, the sequences should be aligned for maximum similarity. “Looped” out sequences from deletions or insertions, or mismatches, are considered differences.) The differences are, suitably, differences or changes at a non-essential residue or a conservative substitution.

In other embodiments, a variant polypeptide includes an amino acid sequence having at least about 50%, 55%, 60%, 65%, 70%, 75%, 80%, 85%, 90%, 91%, 92%, 93%, 94% 95%, 96%, 97%, 98% or more similarity to a corresponding sequence of a FV polypeptide as, for example, set forth in SEQ ID NO: 2, 4, 6, 8, 10 and 12 and has the activity of a snake venom FV polypeptide.

Calculations of sequence similarity or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first Sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In certain embodiments, the percent identity between two amino acid sequences is determined using the Needleman and Wunsch, (1970, J. Mol. Biol. 48: 444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In specific embodiments, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. An non-limiting set of parameters (and the one that should be used unless otherwise specified) includes a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to 53010 nucleic acid molecules of the invention. BLAST protein searches can be performed with the XBLAST program, score=50, wordlength=3 to obtain amino acid sequences homologous to 53010 protein molecules of the invention. To obtain gapped alignments for comparison purposes, Gapped BLAST can be utilized as described in Altschul et al., (1997, Nucleic Acids Res, 25: 3389-3402). When utilizing BLAST and Gapped BLAST programs, the default parameters of the respective programs (e.g., XBLAST and NBLAST) can be used.

Variants of a snake venom FV protein can be identified by screening combinatorial libraries of mutants, e.g., truncation mutants, of a snake venom FV protein. Libraries or fragments e.g., N terminal, C terminal, or internal fragments, of a snake venom protease protein coding sequence can be used to generate a variegated population of fragments for screening and subsequent selection of variants of a snake venom FV protein.

Methods for screening gene products of combinatorial libraries made by point mutation or truncation, and for screening cDNA libraries for gene products having a selected property are known in the art. Such methods are adaptable for rapid screening of the gene libraries generated by combinatorial mutagenesis of snake venom FV proteins.

The FV polypeptides of the invention may be prepared by any suitable procedure known to those of skill in the art. For example, the FV polypeptides may be produced by any convenient method such as by purifying the polypeptide from naturally-occurring reservoirs including snake venom and serum. Methods of purification include lectin (e.g. wheat germ agglutinin) affinity chromatography or separation. The identity and purity of derived FV is determined for example by SDS-polyacrylamide electrophoresis or chromatographically such as by high performance liquid chromatography (HPLC). Alternatively, the FV polypeptides may be synthesized by chemical synthesis, e.g., using solution synthesis or solid phase synthesis as described, for example, in Chapter 9 of Atherton and Shephard (supra) and in Roberge et al., (1995, Science, 269: 202).

Alternatively, the FV polypeptides may be prepared by recombinant techniques. For example, the FV polypeptides of the invention may be prepared by a procedure including the steps of: (a) preparing a construct comprising a polynucleotide sequence that encodes a FV polypeptide and that is operably linked to a regulatory element; (b) introducing the construct into a host cell; (c) culturing the host cell to express the FV polypeptide; and (d) isolating the FV polypeptide from the host cell. In illustrative examples, the nucleotide sequence encodes at least a biologically active portion of the sequences set forth in SEQ ID NO: 2, 4, 6, 8, 10 and 12 or a variant thereof. Recombinant FV polypeptides can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6.

Exemplary nucleotide sequences that encode the FV polypeptides of the invention encompass full-length snake venom FV genes as well as portions of the full-length or substantially full-length nucleotide sequences of the snake venom FV genes or their transcripts or DNA copies of these transcripts. Portions of a snake venom FV nucleotide sequence may encode polypeptide portions or segments that retain the biological activity of the native polypeptide. A portion of a snake venom FV nucleotide sequence that encodes a biologically active fragment of a snake venom FV polypeptide may encode at least about 20, 21, 22, 23, 24, 25, 30, 40, 50, 60, 70, 80, 90, 100, 120, 150, 300 or 400 contiguous amino acid residues, or almost up to the total number of amino acids present in a full-length snake venom FV polypeptide.

The invention also contemplates variants of the snake venom FV nucleotide sequences. Nucleic acid variants can be naturally-occurring, such as allelic variants (same locus), homologs (different locus), and orthologs (different organism) or can be non naturally-occurring. Naturally-occurring variants such as these can be identified with the use of well-known molecular biology techniques, as, for example, with polymerase chain reaction (PCR) and hybridization techniques as known in the art. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). For nucleotide sequences, conservative variants include those sequences that, because of the degeneracy of the genetic code, encode the amino acid sequence of a reference snake venom FV polypeptide. Variant nucleotide sequences also include synthetically derived nucleotide sequences, such as those generated, for example, by using site-directed mutagenesis but which still encode a snake venom FV polypeptide. Generally, variants of a particular snake venom FV nucleotide sequence will have at least about 30%, 40% 50%, 55%, 60%, 65%, 70%, generally at least about 75%, 80%, 85%, desirably about 90% to 95% or more, and more suitably about 98% or more sequence identity to that particular nucleotide sequence as determined by sequence alignment programs described elsewhere herein using default parameters.

Snake venom FV nucleotide sequences can be used to isolate corresponding sequences and alleles from other organisms, particularly other snakes. Methods are readily available in the art for the hybridization of nucleic acid sequences. Coding sequences from other organisms may be isolated according to well known techniques based on their sequence identity with the coding sequences set forth herein. In these techniques all or part of the known coding sequence is used as a probe which selectively hybridizes to other snake venom FV-coding sequences present in a population of cloned genomic DNA fragments or cDNA fragments (i.e., genomic or cDNA libraries) from a chosen organism (e.g., a snake). Accordingly, the present invention also contemplates polynucleotides that hybridize to reference snake venom FV nucleotide sequences, or to their complements, under stringency conditions described below. As used herein, the term “hybridizes under low stringency, medium stringency, high stringency, or very high stringency conditions” describes conditions for hybridization and washing. Guidance for performing hybridization reactions can be found in Ausubel et al., (1998, supra), Sections 6.3.1-6.3.6. Aqueous and non-aqueous methods are described in that reference and either can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6× sodium chloride/sodium citrate (SSC) at about 45° C., followed by two washes in 0.2×SSC, 0.1% SDS at least at 50° C. (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In certain embodiments, a snake venom FV polypeptide is encoded by a polynucleotide that hybridizes to a disclosed nucleotide sequence under very high stringency conditions. One embodiment of very high stringency conditions includes hybridizing 0.5 M sodium phosphate, 7% SDS at 65° C., followed by one or more washes at 0.2×SSC, 1% SDS at 65° C.

Other stringency conditions are well known in the art and a skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8). In general, the Tₘ of a perfectly matched duplex of DNA may be predicted as an approximation by the formula:

Tₘ=81.5+16.6(log₁₀ M)+0.41(% G+C)−0.63(% formamide)−(600/length)

wherein: M is the concentration of Na⁺, preferably in the range of 0.01 molar to 0.4 molar; % G+C is the sum of guanosine and cytosine bases as a percentage of the total number of bases, within the range between 30% and 75% G+C; % formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In one example of a hybridization procedure, a membrane (e.g., a nitrocellulose membrane or a nylon membrane) containing immobilized DNA is hybridized overnight at 42° C. in a hybridization buffer (50% deionized formamide, 5×SSC, 5×Denhardt's solution (0.1% ficoll, 0.1% polyvinylpyrollidone and 0.1% bovine serum albumin), 0.1% SDS and 200 mg/mL denatured salmon sperm DNA) containing labeled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.

The present invention also contemplates the use of snake venom FV chimeric or fusion proteins for treating bleeding episodes or coagulation disorders. As used herein, a snake venom FV “chimeric protein” or “fusion protein” includes a snake venom FV polypeptide linked to a non-snake venom FV polypeptide. A “non-snake venom FV polypeptide” refers to a polypeptide having an amino acid sequence corresponding to a protein which is different from the snake venom FV protein and which is derived from the same or a different organism. The snake venom FV polypeptide of the fusion protein can correspond to all or a portion e.g., a fragment described herein of a snake venom FV amino acid sequence. In a preferred embodiment, a snake venom FV fusion protein includes at least one (or two) biologically active portion of a snake venom FV protein. The non-snake venom FV polypeptide can be fused to the N-terminus or C-terminus of the snake venom FV polypeptide.

The fusion protein can include a moiety which has a high affinity for a ligand. For example, the fusion protein can be a GST-snake venom FV fusion protein in which the snake venom FV sequences are fused to the C-terminus of the GST sequences. Such fusion proteins can facilitate the purification of recombinant snake venom FV. Alternatively, the fusion protein can be a snake venom FV protein containing a heterologous signal sequence at its N-terminus. In certain host cells (e.g., mammalian host cells), expression and/or secretion of snake venom FV can be increased through use of a heterologous signal sequence. In some embodiments, fusion proteins may include all or a part of a serum protein, e.g., an IgG constant region, or human serum albumin.

The snake venom FV fusion proteins of the invention can be incorporated into pharmaceutical compositions and administered to a subject in vivo. The snake venom FV fusion proteins can be used to affect the bioavailability of a snake venom FV substrate.

### 3. FV Pharmaceutical Compositions and their Uses

The present invention also contemplates the use of the snake venom FV polypeptides as described herein in compositions and methods for treating bleeding episodes or coagulation disorders. Accordingly, the FV polypeptides of the present invention are suitably administered in a pharmaceutical composition, comprising a pharmaceutically acceptable carrier. The pharmaceutically acceptable carriers may be selected from a non limiting group including sugars, starches, cellulose and its derivatives, malt, gelatine, talc, calcium sulphate, vegetable oils, synthetic oils, polyols, alginic acid, phosphate buffered solutions, emulsifiers, polyethylene glycol and different molecular weights thereof, isotonic saline and salts such as mineral acid salts including hydrochlorides, bromides and sulphates, organic acids such as acetates, propionates and malonates and pyrogen-free water. A variety of aqueous carriers can be used, such as water, buffered water, 0.4% saline, 0.3% glycine and the like. The compositions of the invention can also be formulated using non-aqueous carriers, such as for example, in the form of a gel or as liposome preparations for delivery or targeting to the sites of injury. Liposome preparations are generally described in for example, U.S. Pat. Nos. 4,837,028, 4,501,728 and 4,975,282.

A useful reference describing pharmaceutically acceptable carriers, diluents and excipients is Remington's Pharmaceutical Sciences (Mack Publishing Co. N.J. USA, 1991) which is incorporated herein by reference. Supplementary active compounds can also be incorporated into the compositions.

The pharmaceutical compositions of the invention can be used to promote or otherwise facilitate blood coagulation. Examples of use include administration to bleeding wounds such as during surgery or following injury or trauma.

In some embodiments, a snake venom FV polypeptide is the only blood-coagulating component present in the pharmaceutical composition. In this regard, the present inventors have found that pharmaceutical compositions, which comprise various embodiments of a snake venom FV polypeptide of the invention (e.g., a polypeptide which comprises the sequence set forth in SEQ ID NO:2 and processed forms thereof), coagulates blood rapidly without a need for the sequential or combinatorial action of plural components such as co-factors, snake venom FXa, or other blood clotting factors such as FVII or FVIIa.

In some embodiments, the pharmaceutical composition may contain other components, including without limitation, pH adjusting and buffering agents and/or tonicity adjusting agents, such as, for example, sodium acetate, sodium lactate, sodium chloride, potassium chloride and calcium chloride.

The pharmaceutical composition can be formulated to promote stability of the snake venom FV polypeptide, e.g., to reduce digestion, such as autodigestion, of the snake venom FV. The stability of the snake venom FV can be promoted, for example, by preparing and/or providing the snake venom FV in a pharmaceutical composition having a pH of about 5 to 9, or about 6.5 to 7. The stability of the snake venom FV can also be stabilized by providing the snake venom FV in a pharmaceutical composition further includes, e.g., a stabilizer, such as a polyol. In such embodiments, the pharmaceutical composition can include about 5%, 10%, 20% or more of a polyol (or polyols). An example of a polyol which can be used in the pharmaceutical composition is glycerol. In other aspects, the stability of the snake venom FV can be increased by providing the snake venom FV in a crystallized, freeze-dried or lyophilized form. If the composition is frozen, the composition should be thawed prior to the time of use. In another embodiment, the invention features a composition which includes a snake venom FV, e.g., a snake venom FV described herein, and which has a pH of about 5 to 9, or about 6.5 to 7. The invention also features a composition which includes a snake venom FV, e.g., a snake venom FV described herein, and a stabilizing agent, e.g., a polyol, e.g., glycerol. The polyol can be present at about for example 5%, 10% or 20%.

The dosage of the composition comprising an FV polypeptide of the invention depends upon the particular use of that polypeptide, but the dosage should be an effective amount for the composition to perform its intended use. Data obtained from cell culture assays and animal studies can be used in formulating a range of dosage levels for use in humans. Generally, for a composition comprising a snake venom FV polypeptide that is an aqueous solution, it is believed that from about 1 to 100 nM of such a composition is sufficient to increase fibrin clot formation. However, depending on the use of the composition, the dosage can range from about 20 nM to 5 μM. For example, FV polypeptides of the invention are suitably administered to a subject, e.g., a human or a non-human mammal, such as a domestic animal, generally at dosages of at least about 0.1 to about 1 mg/kg given intravenously, although other dosages may provide beneficial results.

In some embodiments, pharmaceutical compositions of the invention are topically administered to a wound, surgical incision or other location where blood loss is to be prevented. To this end, bandages, patches, gauze, surgical tape, cotton swabs or other absorbent materials or supportive matrices may be coated, impregnated or chemically bonded with a composition which includes a snake venom FV of the invention for topical administration. Topical administration is desirable in these applications. In addition, sutures and staples coated or chemically bonded with a composition which includes a snake venom FV can be used.

In the case of internal bleeding or for systemic bleeding, the composition may be injected intravenously, subcutaneously, intradermally or intramuscularly and can be administered as a single or multiple dose. The specific dose to be administered in therapy can be determined by a physician and will depend on the route of administration and on the weight and condition of the subject.

The pharmaceutical compositions can be included in a container, pack, or dispenser together with instructions for administration. Accordingly, the present invention also contemplates kits for preventing or reducing blood loss or bleeding in a subject. These kits will generally comprise a snake venom FV polypeptide as broadly described herein and one or more other elements such as for example: instructions for use; other reagents and/or other therapeutic agents (e.g., one or more of: an anti-microbial, e.g., an antibiotic, an antiviral, an antifungal, an antiparasitic agent, an anti-inflammatory agent, an antihistamine, an anti-fibrolytic agent, an analgesic, and a growth factor); a diluent; devices, e.g., containers, e.g., sterile containers, or other materials for preparing the snake venom FV for administration; pharmaceutically acceptable carriers (e.g., a stabilizer); and devices or other materials for administration to a subject (e.g., syringes, applicators, bandages, spray or aerosol devices). The instructions can include instructions for therapeutic application including suggested dosages and/or modes of administration, e.g., in a subject with external and/or internal bleeding. In other applications, the snake venom FV can be administered in combination with other components, and the kit can include instructions on the amount, dosage, and timing of administration of the snake venom FV and the other components.

In some embodiments, the snake venom FV may be supplied in lyophilized or freeze dried form. In such embodiments, the kit can include one or more of: instructions for thawing and/or hydrolyzing, and a pharmaceutically acceptable carrier or diluent. In some embodiments, the kit can include instructions for a diluent or a pre-measured amount of a diluent.

The present invention also encompasses methods for preventing or reducing blood loss or bleeding in a subject. The method can include: administering a snake venom FV polypeptide to a desired site in a subject in an amount effective to promote or increase blood clot formation, and to thereby increase clotting and/or decrease blood or fluid loss. The compositions can be applied directly to the wound, other tissue or other desired site. Typically for external wounds it can be applied directly by any means, including spraying the wound. It can also be applied internally, such as during a surgical procedure or through injection.

In some embodiments, the subject is a mammal, e.g., a human. Since the snake venom FV molecules described herein are not typically from blood, concerns regarding the risk of blood born pathogens or other infectious agents which can be found in clotting agents obtained from components of blood are alleviated.

The methods, kits or pharmaceutical compositions of the invention can be used, e.g., for stopping or reducing bleeding, preventing or inhibiting bleeding, healing wounds, and/or sealing a wound. The methods, kits and pharmaceutical compositions can be used in various surgical settings including: surgery of the nervous system; surgery of the nose, mouth or pharynx; surgery of the respiratory system; surgery of the cardiovascular system; surgery of hemic or lymphatic systems; surgery of the digestive system; surgery of the urinary system; surgery of the reproductive system; surgery of the musculoskeletal system; surgery of the integumentary system; plastic surgery; orthopedic surgery, and transplant surgery. For example, the snake venom FV can be used in vascular surgery including providing hemostasis for stitch hole bleeding of distal coronary artery anastomoses; left ventricular suture lines; aortotomy and cannulation sites; diffuse epimyocardial bleeding seen in reoperations; and oozing from venous bleeding sites, e.g. at atrial, caval, or right ventricular levels. The subject invention is also useful for sealing of dacron artery grafts prior to grafting, sealing tissues outside the body, stopping bleeding from damaged spleens (thereby saving the organ), livers, and other parenchymatous organs; sealing tracheal and bronchial anastomoses and air leaks or lacerations of the lung, sealing bronchial stumps, bronchial fistulas and esophageal fistulas; and for sutureless seamless healing (“Zipper” technique). The subject invention is further useful for providing hemostasis in corneal transplants, nosebleeds, post tonsillectomies, teeth extractions and other applications. See G. F. Gestring and R. Lermer, Vascular Surgery, 294-304, September/October 1983. Also, the pharmaceutical compositions of the invention are ° especially suited for individuals with coagulation defects such as for example hemophilia (e.g., Hemophilia A and Hemophilia B).

As discussed above, the snake venom FV polypeptide may be formulated as part of a wound dressing, bandage, patch, gauze, surgical tape, cotton swabs or other absorbent materials or supportive matrices. The dressing and bandage are easy-to-use, requiring no advanced technical knowledge or skill to operate. They can even be self-administered as an emergency first aid measure. Such wound dressings and bandages can be used in various field applications, such as in trauma packs for soldiers, rescue workers, ambulance/paramedic teams, firemen, and in early trauma and first aid treatment by emergency room personnel in hospitals and clinics, particularly in disaster situations. Such dressings may also have utility in first aid kits for use by the general public or by medical practitioners. The snake venom FV containing wound dressing or bandage can further include one or more of a stabilizing agent, or other compound or agent such as those described herein. For example, the wound dressing or bandage can further include: an analgesic, an antiviral, an antifungal, an antiparasitic agent, an anti-inflammatory agent, an antihistamine, an anti-fibrolytic agent, and a growth factor.

More than one compound other than the snake venom FV polypeptide can be added to the composition, to be released simultaneously, or each can be released in a predetermined time-release manner. The additional compound (or compounds) added to the composition can be added at a concentration such that it will be effective for its intended purpose, e.g., an antibiotic will inhibit the growth of microbes, an analgesic will relieve pain, etc. In some embodiments, the dressing or bandage can include an adhesive layer and/or backing layer. The backing of the dressing or bandage may be of conventional, non-reabsorbable materials, e.g., a silicone patch or plastic material; or it may be of biocompatible, resorbable materials, e.g., chitin or its derivatives.

For other applications, such as for use in surgery or as an internal clotting factor, the snake venom FV may be formulated to be administered either by injection or in some form of internal application.

Although the speed with which the composition forms clots may be to some degree dictated by the application, e.g., rapid setting for arterial wounds and hemorrhaging tissue damage, slower setting for treatment of wounds to bony tissue. In specific embodiments, clotting is evident within one minute after application when the reagent is applied topically and within five minutes when the reagent is injected intravenously. The latter time allows for equilibration in blood.

In other embodiments, snake venom FV polypeptides can be used either by themselves or in combination with other agents to produce serum in vitro.

In order that the invention may be readily understood and put into practical effect, particular preferred embodiments will now be described by way of the following non-limiting examples.

### EXAMPLES

### Example 1

### Isolation of P. textilis FV from Venom

The prothrombin activator complex was isolated from P. textilis venom as described in the inventors earlier work Masci et al., (1988, Biochem Int, 17: 825-835) incorporated herein by reference. 4 mg/mL of prothrombin activator was stored in 50% glycerol at −20° C. Sephacryl S-300 was obtained from Amersham Pharmacia Biotech., Uppsala, Sweden, and the synthetic chromogenic substrate S-2222 was obtained from Chromogenex, Stockholm, Sweden. Outdated citrated plasma was obtained from normal, virus-screened volunteers made available by Princess Alexandra Hospital Blood Bank. Hampton 1 and 2 screen kits were obtained from Hampton Research, United States of America. Wizard 1 and 2 screen kits were obtained from Emerald Biostructures, United Kingdom.

The first step in the purification of P. textilis-snake venom FV was to isolate Brown snake venom protease complex from crude venom, as described in Masci et al., (1988, Supra). Con A-Sepharose 4B was packed into a 2.5×16 cm column, washed as recommended by the manufacturer and equilibrated (with starting buffer (0.05 M Tris-HC1, pH 7.4). P. textilis venom (233 mg dry weight) was reconstituted in 10 ml starting buffer and placed into a 37° C. water bath until dissolved. The sample was loaded onto the column and washed with column buffers until the baseline returned to zero. Elution buffer (0.02 M methyl a-D mannopyranoside in 0.05 M Tris-HCl) was applied to the column to elute bound protein (Brown snake venom protease complex) from the Con A-Sepharose 4B. The flow rate of the column was 52 ml/hour. The UV dual wavelength detector was set at 280 mm with attenuations of 0.32 and 0.64 absorbance units full scale (AUFS). Fractions with S-2222 hydrolytic activity were pooled and concentrated in an Amicon concentrator, model 405, with a YM3 membrane, having a flow rate of 48 mL/hour. Purified Brown snake venom protease complex was stored in 50% glycerol at −20° C.

Treatment with sodium isothiocyanate dissociated the complex, allowing separation of the FV-like component by gel chromatography on Superdex 75. The FV-like component was then treated with the irreversible FXa inhibitor DDACK, see Speijer et al., (1986, J Biol Chem, 13258-13267) to inhibit trace amounts of FXa activity which may have been present in the purified snake venom FV. Complete inhibition was confirmed by assays using the FXa chromogenic substrate S-2222 and by plasma clotting assay.

Residual snake FXa protein was removed by affinity adsorption onto Sepharose beads derivatized with polyclonal antibody against snake FXa heavy chain.

### Example 2

### Clotting of Citrated Plasma with P. textilis Venom Fv

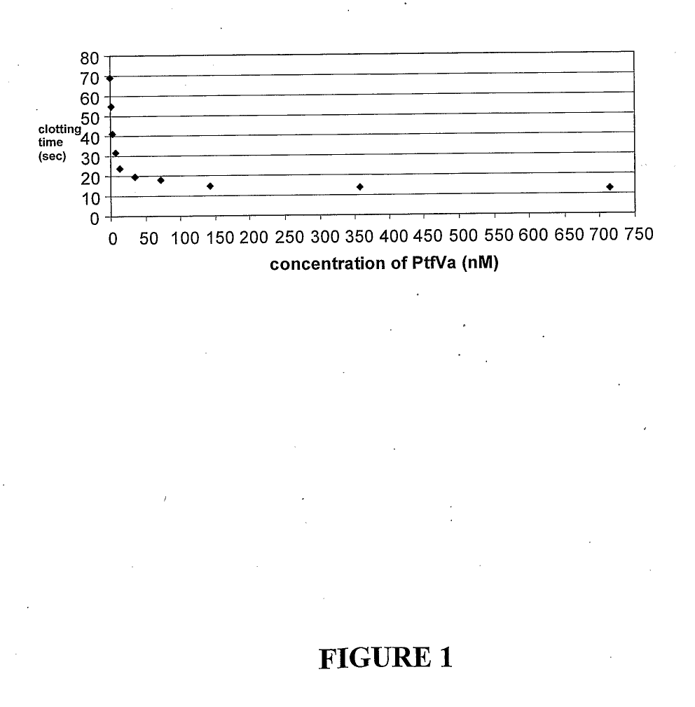

The clotting time for re-calcified citrated plasma was determined in the presence and absence of snake venom FV, wherein even at low nanomolar concentrations a big increase in the rate of clotting of citrated plasma was accomplished. The simple explanation of this effect is the formation of a highly active hybrid prothrombinase complex between the added snake venom FV and human FXa. Table 1 below illustrates clotting times for citrated plasma determined using a Hyland-Clotek machine at 37° C. The reaction mixture (350 μL volume), consisted of 100 μL citrated plasma; 100 μl, of tris buffered saline; 50 μL of 0.2 M calcium; 50 μL platelin LS (phospholipid) and; 50 μL of snake venom FV preparation or buffer. Table 2 on page 59 demonstrates citrated plasma clotting in the presence of calcium (Ca), phospholipid (PL) and P. textilis snake venom FV and FIG. 1 provides an example of a graph illustrating the clotting time of recalcified citrated plasma in the presence of phospholipid and P. textilis venom FV.

TABLE 1

| | Citrated Plasma Clotting |
|---|---|
| FV (nM) | Time (Sec) |
| 0 | 151 |
| 700 | 13 |
| 70 | 18 |
| 7 | 32 |
| 0.7 | 69 |

### Example 3

### Clotting of Whole Blood

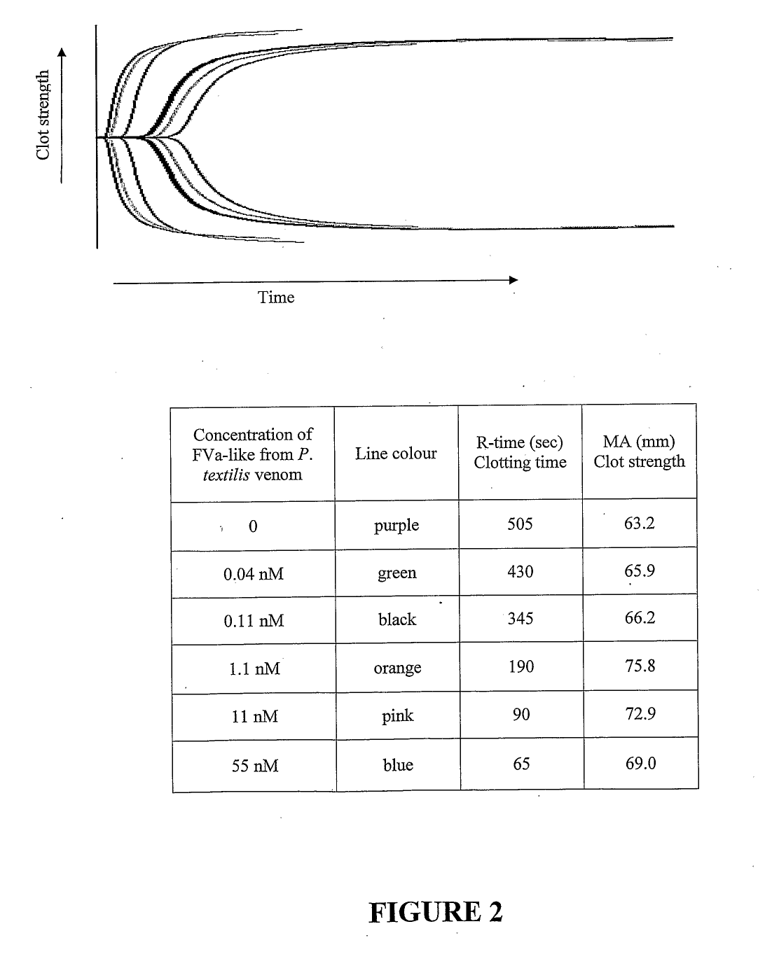

The effect of added snake venom FV on the clotting of re-calcified citrated human blood was determined using a Hemoscope thromboelastograph (TEG) (see FIG. 2). FIG. 1 confirms the large enhancement of clotting caused by the addition of small amounts of snake venom FV and, like the plasma clotting results, are consistent with the formation of hybrid snake venom FVa-human FXa complex.

TABLE 2

| | Citrated | | Fva | PL | Ca | Clotting time | Clotting time | CT (ave) |
|---|---|---|---|---|---|---|---|---|
| # | plasma | Buffer | A280 = 1.080 | Platelin LS | (0.2M) | (sec) (1) | (sec) (2) | (sec) |
| 1 | 100 | 150 | 0 | 50 | 50 | 152.1 | 150.5 | 150.3 |
| 2 | 100 | 150 | 50 | 50 | 0 | >300 | >300 | >300 |
| 3 | 100 | 150 | 50 | 0 | 50 | 54.1 | 54.5 | 54.3 |
| 4 | 100 | 150 | 50 (1/10) | 0 | 50 | 68.9 | 67.0 | 67.5 |
| 5 | 100 | 150 | 50 (1/100) | 0 | 50 | 84.3 | 97.4 | 90.8 |
| 6 | 100 | 150 | 50 (1/1000) | 0 | 50 | >300 | >300 | >300 |
| 7 | 100 | 100 | 50 | 50 | 50 | 12.6 | 12.8 | 12.7 |
| 8 | 100 | 125 | 25 | 50 | 50 | 13.3 | 13.8 | 13.6 |
| 9 | 100 | 140 | 10 | 50 | 50 | 15.2 | 14.8 | 15.0 |
| 10 | 100 | 100 | 50 (1/10) | 50 | 50 | 18.3 | 17.6 | 18.0 |
| 11 | 100 | 125 | 25 (1/10) | 50 | 50 | 19.6 | 19.8 | 19.7 |
| 12 | 100 | 140 | 10 (1/10) | 50 | 50 | 24.1 | 23.4 | 23.7 |
| 13 | 100 | 100 | 50 (1/100) | 50 | 50 | 32.3 | 31.0 | 31.6 |
| 14 | 100 | 125 | 25 (1/100) | 50 | 50 | 39.5 | 42.5 | 41.0 |
| 15 | 100 | 140 | 10 (1/100) | 50 | 50 | 53.3 | 56.0 | 54.7 |
| 16 | 100 | 100 | 50 (1/1000) | 50 | 50 | 69.7 | 67.7 | 68.7 |

The inclusion of 20 nM of APC in the clotting mixture (for clotting of re-calcified citrated whole blood) increased the clotting time from 505 seconds to 1333 seconds and weakened the clot strength from 12 to 7 kdyn/cm2. Co-addition of 280 nM of snake venom FV with the APC resulted in a clotting time of 127 seconds and restored the clot strength. These results are readily explained if the hybrid snake-human complex is much more stable than the human Va-Xa complex towards APC cleavage.

### Example 4

### Mouse Tail Excision Bleeding Model

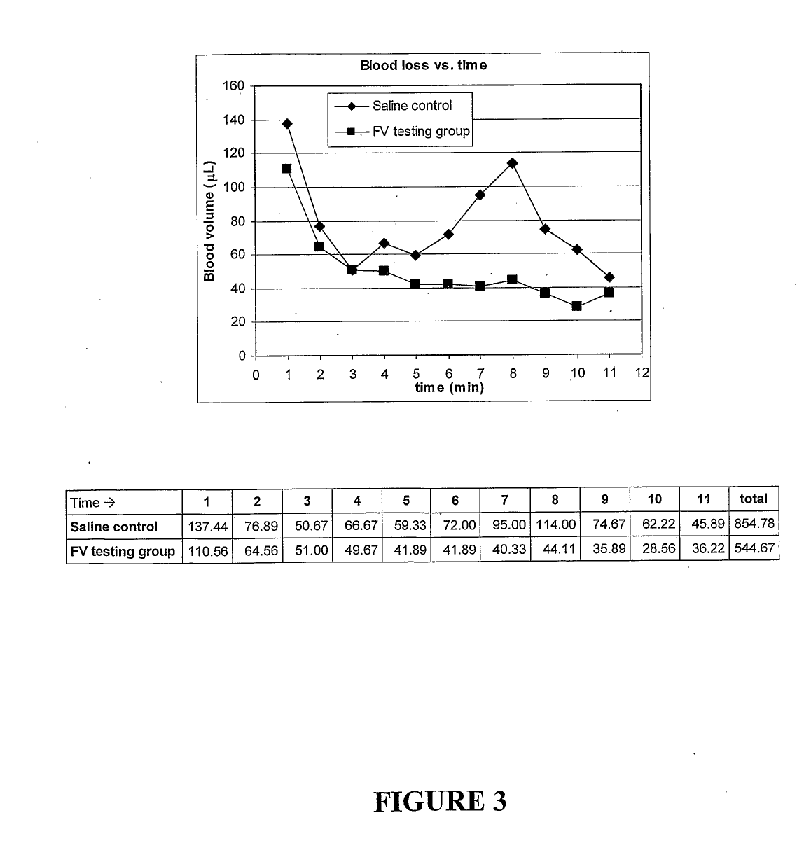

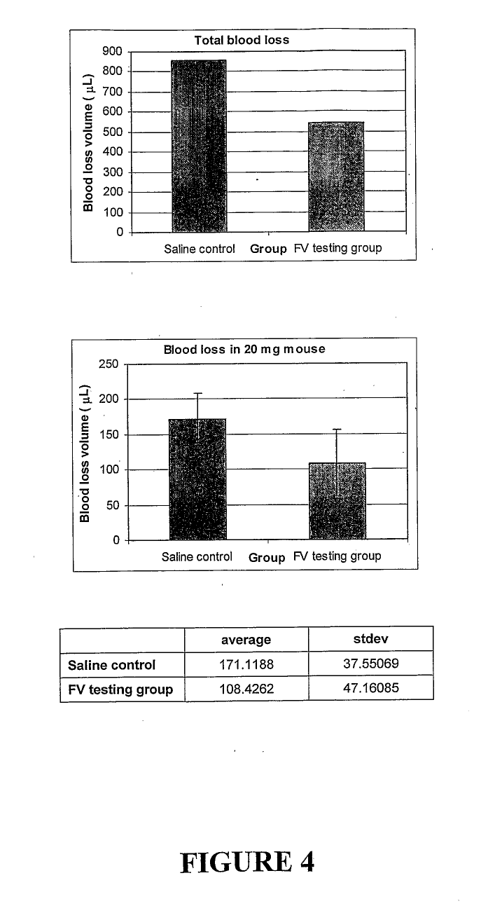

The protocol used was adapted from that of Tanabe et al., (1999, Thromb Haemos, 8: 828-836) herein incorporated by reference. Adult B57/BL mice (20-30 g) were anaesthetized (isoflurane, 2.5%, 1.5 L/min) and the final 10 mm of the tail was surgically excised with a sterile blade. The proximal tail surface was immediately immersed in saline (100 mL) with and without P. textilis FV (50 nmol/L) for 1 min. Blood lost from the cut surface was then adsorbed onto a filter paper (Whatman No. 54 filter paper) chad (1 cm diameter) which was replaced at 1 min intervals. Hemoglobin on the chads and in the initial saline solutions was measured by the method of Shaw et al., (1972, Contracept, 5: 497-513). FIGS. 3 and 4 provide illustrative examples of blood loss tests run using the mouse excision bleeding model. Ethical approval was obtained from the Animal Care Committee of the Queensland Institute of Medical Research and the University of Queensland Animals Ethics Committees and protocols were compliant with NHMRC AEEC guidelines.

### Western Blot

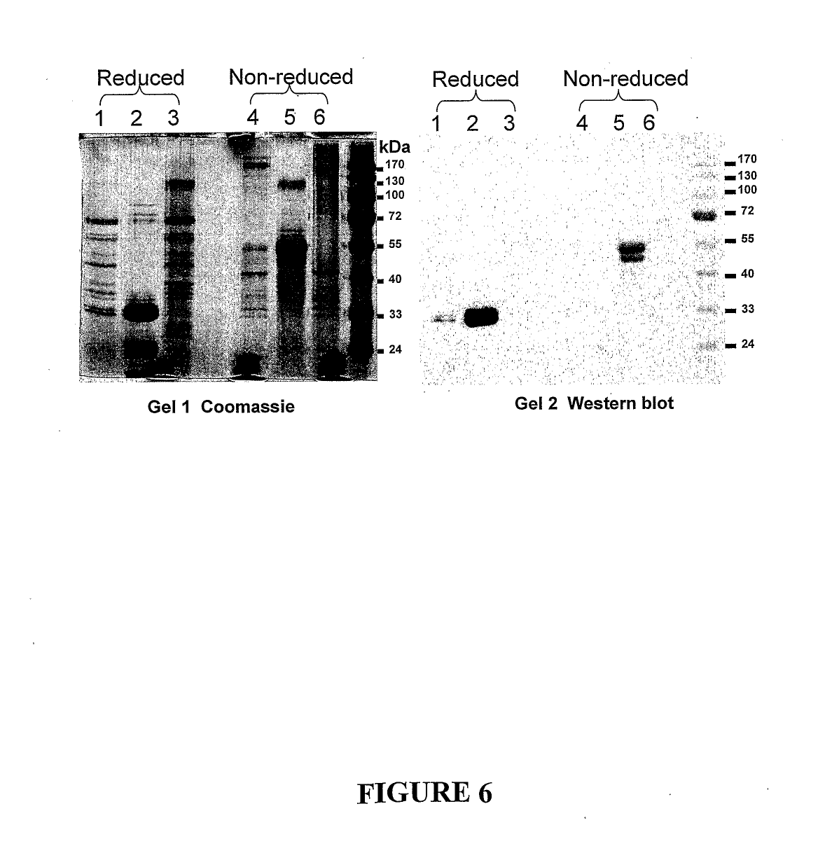

The results of SDS PAGE and Western blot analysis of reduced and non-reduced preparations of prothrombin activator complex isolated from P. textilis venom; isolated FXa like protease from P. textilis and; FVa isolated from P. textilis venom demonstrated that with the anti-protease heavy chain antibody (sheep antiserum against recombinant GST fusion protein with the heavy chain of P. textilis FXa like protease), the FV preparation appears depleted of FXa (see FIG. 6). The left hand gel illustrated in FIG. 6 is the result of Coomassie staining and the right hand figure is a Western blot from the same gel.

### Example 5

### Intravenous Bleeding Data

The protocol used was similar to the mouse tail excision bleeding model outlined in paragraph [0269]. Adult B57/BL mice (20-30 g) were anaesthetized (isoflurane, 2.5%, 1.5 L/min) and intravenously injected with either 100 μL of 500 nmol/L FV in saline; 100 μL of saline (control); or 100 μL of Aprotinin in saline (110 μmol/L). There were ten animals in each group. After 3 minutes of allowing the intravenously injected solution to equilibrate in the blood stream, the final 10 mm of the tail was surgically excised with a sterile blade. Blood lost from the cut surface was then adsorbed onto a filter paper (Whatman No. 54 filter paper) chad (1 cm diameter) which was replaced at 1 min intervals. Hemoglobin on the chads was measured by the method of Shaw et al., (1972, Contracept, 5: 497-513).

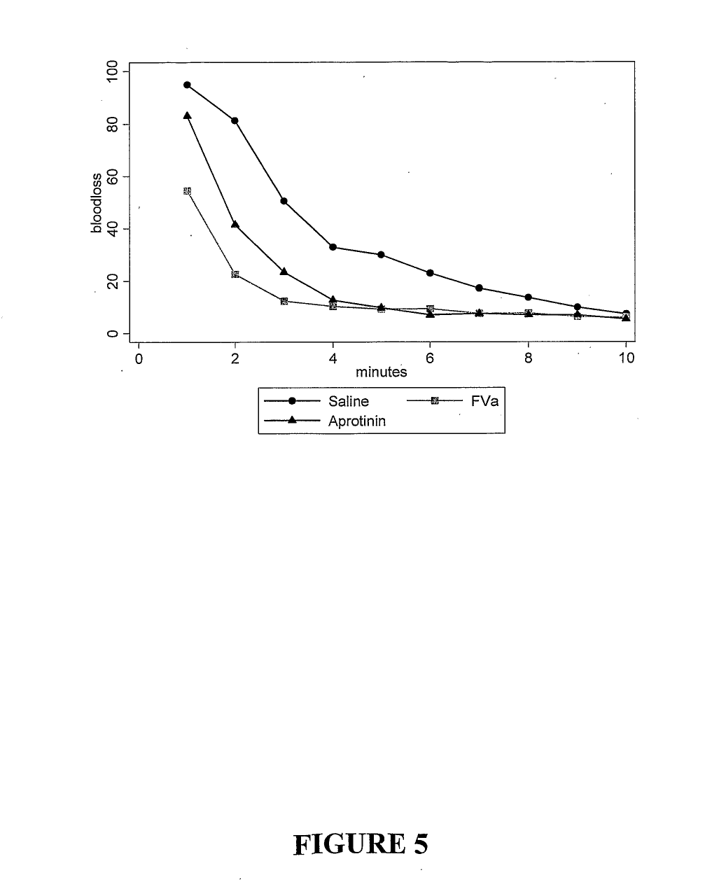

FIG. 5 provides an illustrative example of blood loss tests run using the intravenous mouse excision bleeding model. Ethical approval was obtained from the Animal Care Committee of the Queensland Institute of Medical Research and the University of Queensland Animals Ethics Committees and protocols were compliant with NHMRC AEEC guidelines.

Statistical analysis of the results using ANOVA demonstrated an interaction between treatment group and time in minutes (p=0.041). This is illustrated in the FIG. 5 where the lines for FV and Aprotinin cross over at the 5 minute time-point. There was strong evidence of a treatment effect (p<0.0001) and a time effect (p<0.0001): Multiple comparisons showed that both experimental groups (FV and aprotinin) had lower average blood loss compared to control (p<0.05). On average blood loss was 58% (95% confidence interval: 47% to 66%) lower in the aprotinin group compared to the control group and 56% (95% confidence interval: 45% to 65%) lower in the FV group compared to the control group. Blood loss was also lower on average at all subsequent time points in comparison to blood loss at 1 minute (p<0.05). Table 3 below illustrates the average blood loss by time in minutes for each treatment group and the P-values relate to the Wilcoxon 2-sample test.

TABLE 3

| | | | | | | | Signifi- |
|---|---|---|---|---|---|---|---|
| | Saline | | FVa | | Aprotinin | | cance |
| | (n = 10) | | (n = 10) | | (n = 10) | | (P-value)* |
| Time | μL | | μL | | μL | | F vs S |
| (min) | blood | SD | blood | SD | blood | SD | A vs S |
| 1 | 94.88 | 37.49 | 54.54 | 43.36 | 83.06 | 51.44 | 0.059 |
| | | | | | | | 0.25 |
| 2 | 81.20 | 50.81 | 22.59 | 14.44 | 41.47 | 16.14 | 0.0046 |
| | | | | | | | 0.059 |
| 3 | 50.43 | 26.84 | 12.27 | 5.54 | 23.39 | 10.91 | 0.0014 |
| | | | | | | | 0.023 |
| 4 | 32.91 | 13.50 | 10.20 | 3.73 | 12.66 | 7.17 | 0.0014 |
| | | | | | | | 0.0027 |
| 5 | 29.89 | 15.35 | 9.20 | 3.22 | 9.64 | 5.37 | 0.0016 |
| | | | | | | | 0.0023 |
| 6 | 23.00 | 11.15 | 9.28 | 3.70 | 6.93 | 5.05 | 0.0054 |
| | | | | | | | 0.0046 |
| 7 | 17.16 | 9.46 | 7.49 | 2.29 | 7.45 | 6.23 | 0.023 |
| | | | | | | | 0.051 |
| 8 | 13.56 | 10.55 | 7.69 | 3.84 | 6.95 | 5.60 | 0.17 |
| | | | | | | | 0.13 |
| 9 | 9.83 | 7.86 | 6.16 | 3.16 | 6.90 | 5.91 | 0.35 |
| | | | | | | | 0.43 |
| 10 | 7.26 | 4.57 | 5.95 | 3.36 | 5.29 | 3.97 | 0.52 |
| | | | | | | | 0.35 |
| TOTAL | 360.15 | 148.32 | 145.40 | 59.06 | 203.78 | 77.87 | |

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1.-48. (canceled)

49. A pharmaceutical composition for treating blood loss or coagulation disorders, comprising a snake venom FV polypeptide and a pharmaceutically acceptable carrier or diluent, wherein the composition excludes a snake venom FXa polypeptide, and wherein the snake venom FV polypeptide comprises an amino acid sequence selected from the group consisting of: (a) an amino acid sequence that shares at least 90% sequence identity with the sequence set forth in any one of SEQ ID NO: 2, 4, 6, 8, 10 or 12; or (b) an amino acid sequence which is encoded by a nucleotide sequence that shares at least 90% sequence identity with the sequence set forth in any one of SEQ ID NO: 1, 3, 5, 7, 9 or 11; or (c) an amino acid sequence which is encoded by a nucleotide sequence that hybridizes under high stringency conditions to a complement of the sequence set forth in any one of SEQ ID NO: 1, 3, 5, 7, 9 or 11, wherein the amino acid sequence of (a), (b) or (c) has any one or more activity selected from the group consisting of: bleeding-inhibiting activity, clotting time-reducing activity; haemostasis-enhancing activity; clot lysis time prolonging activity; and clot strength-increasing activity.

50. A composition according to claim 49, which is formulated for topical administration.

51. A composition according to claim 49, which is formulated for intravenous administration.

52. A composition according to claim 49, which excludes FVII and/or FVIIa.

53. A composition according to claim 49, wherein the snake venom FV polypeptide comprises a light chain and a heavy chain domain, as shown in FIG. 7.

54. A composition according to claim 49, wherein an activation peptide domain is interposed between the light chain and heavy chain domains, as shown in FIG. 7.

55. A composition according to claim 49, wherein the snake venom FV polypeptides lacks an activation peptide domain.

56. A composition according to claim 49, wherein the snake venom FV polypeptide lacks at least one of the signal peptide domain and the activation peptide domain.

57. A composition according to claim 49, wherein the snake venom FV polypeptide comprises an Activated Protein C (APC) site at residues 818-819 and/or residues 537-538 relative to the consensus numbering of FIG. 7.

58. A composition according to claim 49, wherein the snake venom FV polypeptide has one or more fewer APC sites than a wild-type mammalian FV.

59. A kit comprising a composition according to claim 49 and instructions for using the kit to treat or prevent a bleeding episode or a coagulation disorder in a subject.

60. A kit according to claim 59, wherein the snake venom FV polypeptide is formulated for administration in an amount that is effective for achieving any one or more of the following: (1) inhibition of bleeding; (2) reduction of clotting time; (3) enhancing hemostasis; (4) prolonging clot lysis time; and (5) increase in clot strength.

61. A kit according to claim 59, wherein the snake venom FV polypeptide is formulated for administration by a person other than the subject.

62. A kit according to claim 59, wherein the snake venom FV polypeptide is formulated for self administration.

63. A kit according to claim 59, wherein the snake venom FV polypeptide is formulated for provision to the subject in advance of a need to use it.

64. A method for treating or preventing a bleeding episode or a coagulation disorder in a subject, the method comprising administering to the subject a bleeding-inhibiting effective amount of a composition according to claim 49.
